(19) United States
(12) Patent Application Publication
(10) Pub. No.: US 2025/0268167 A1
(43) Pub. Date: Aug. 28, 2025

(54) LETTUCE VARIETY 'PS 1541'

(71) Applicant: Pinnacle Seed, Inc., Carmel, CA (US)

(72) Inventors: Mike VANOLI, Carmel, CA (US); Michael KODA, Carmel, CA (US)

(73) Assignee: Pinnacle Seed, Inc., Carmel, CA (US)

(21) Appl. No.: 19/058,615

(22) Filed: Feb. 20, 2025

Related U.S. Application Data
(60) Provisional application No. 63/556,788, filed on Feb. 22, 2024.

(51) Int. Cl. A01H 6/14; A01H 5/12

(52) U.S. Cl. CPC A01H 6/1472; A01H 5/12

(57) ABSTRACT

New lettuce variety designated ‘PS 1541’ is described. ‘PS 1541’ exhibits stability and uniformity.

### CROSS-REFERENCE TO RELATED APPLICATION

This application claims the benefit of U.S. Provisional Application No. 63/556,788, filed Feb. 22, 2024, which is hereby incorporated by reference in its entirety.

### FIELD

This invention relates to the field of plant breeding. In particular, this invention relates to new lettuce, Lactuca sativa, varieties, ‘PS 1540’, ‘PS 1541’, ‘PS 1546’, and ‘PS 1902’.

### BACKGROUND

Lettuce is an increasingly popular crop. Worldwide lettuce consumption continues to increase. As a result of this demand, there is a continued need for new lettuce varieties. In particular, there is a need for improved iceberg, romaine, and green leaf lettuce varieties that exhibit desirable market qualities, improved growth habits, bolting and tip burn tolerance, and disease resistance.

### SUMMARY

In order to meet these needs, the present invention is directed to improved lettuce varieties.

In one embodiment, the present invention is directed to lettuce, Lactuca sativa, seed designated as ‘PS 1540’ having ATCC Accession Number X1. In one embodiment, the present invention is directed to a Lactuca sativa lettuce plant and parts isolated therefrom produced by growing ‘PS 1540’ lettuce seed. In another embodiment, the present invention is directed to a Lactuca sativa plant and parts isolated therefrom having all the physiological and morphological characteristics of a Lactuca sativa plant produced by growing ‘PS 1540’ lettuce seed having ATCC Accession Number X1. In still another embodiment, the present invention is directed to an F₁ hybrid Lactuca sativa lettuce seed, plants grown from the seed, and a head isolated therefrom having ‘PS 1540’ as a parent, where ‘PS 1540’ is grown from ‘PS 1540’ lettuce seed having ATCC Accession Number X1.

Lettuce plant parts include lettuce heads, lettuce leaves, parts of lettuce leaves, pollen, ovules, flowers, and the like. In another embodiment, the present invention is further directed to lettuce heads, lettuce leaves, parts of lettuce leaves, flowers, pollen, and ovules isolated from ‘PS 1540’ lettuce plants. In another embodiment, the present invention is further directed to tissue or cell culture of ‘PS 1540’ lettuce plants, and to lettuce plants regenerated from the tissue or cell culture, where the plant has all of the morphological and physiological characteristics of ‘PS 1540’ lettuce plants. In another embodiment, the present invention is further directed to tissue cell culture produced from protoplasts or cells of a lettuce plant, wherein the lettuce plant is produced by growing ‘PS 1540’ lettuce seed having ATCC Accession Number X1.

The present invention is further directed to a method of selecting lettuce plants by: a) growing more than one ‘PS 1540’ lettuce plant, where the plants are grown from lettuce seed having ATCC Accession Number X1; and b) selecting a plant from step a). The present invention is further directed to lettuce plants and seeds produced therefrom, where the lettuce plants and seeds are isolated by the selection method of the invention.

In another embodiment, the present invention is further directed to a method of breeding lettuce plants by crossing a lettuce plant with a plant grown from ‘PS 1540’ lettuce seed having ATCC Accession Number X1. In still another embodiment, the present invention is further directed to lettuce plants, lettuce parts from the lettuce plants (e.g., lettuce heads), and seeds produced therefrom where the lettuce plant is isolated by the breeding method of the invention.

In one embodiment, the present invention is directed to lettuce, Lactuca sativa, seed designated as ‘PS 1541’ having ATCC Accession Number X2. In one embodiment, the present invention is directed to a Lactuca sativa lettuce plant and parts isolated therefrom produced by growing ‘PS 1541’ lettuce seed. In another embodiment, the present invention is directed to a Lactuca sativa plant and parts isolated therefrom having all the physiological and morphological characteristics of a Lactuca sativa plant produced by growing ‘PS 1541’ lettuce seed having ATCC Accession Number X2. In still another embodiment, the present invention is directed to an F₁ hybrid Lactuca sativa lettuce seed, plants grown from the seed, and a head isolated therefrom having ‘PS 1541’ as a parent, where ‘PS 1541’ is grown from ‘PS 1541’ lettuce seed having ATCC Accession Number X2.

Lettuce plant parts include lettuce heads, lettuce leaves, parts of lettuce leaves, pollen, ovules, flowers, and the like. In another embodiment, the present invention is further directed to lettuce heads, lettuce leaves, parts of lettuce leaves, flowers, pollen, and ovules isolated from ‘PS 1541’ lettuce plants. In another embodiment, the present invention is further directed to tissue or cell culture of ‘PS 1541’ lettuce plants, and to lettuce plants regenerated from the tissue or cell culture, where the plant has all of the morphological and physiological characteristics of ‘PS 1541’ lettuce plants. In another embodiment, the present invention is further directed to tissue cell culture produced from protoplasts or cells of a lettuce plant, wherein the lettuce plant is produced by growing ‘PS 1541’ lettuce seed having ATCC Accession Number X2.

The present invention is further directed to a method of selecting lettuce plants by: a) growing more than one ‘PS 1541’ lettuce plant, where the plants are grown from lettuce seed having ATCC Accession Number X2; and b) selecting a plant from step a). The present invention is further directed to lettuce plants and seeds produced therefrom, where the lettuce plants and seeds are isolated by the selection method of the invention.

In another embodiment, the present invention is further directed to a method of breeding lettuce plants by crossing a lettuce plant with a plant grown from ‘PS 1541’ lettuce seed having ATCC Accession Number X2. In still another embodiment, the present invention is further directed to lettuce plants, lettuce parts from the lettuce plants (e.g., lettuce heads), and seeds produced therefrom where the lettuce plant is isolated by the breeding method of the invention.

In one embodiment, the present invention is directed to lettuce, Lactuca sativa, seed designated as ‘PS 1546’ having ATCC Accession Number X4. In one embodiment, the present invention is directed to a Lactuca sativa lettuce plant and parts isolated therefrom produced by growing ‘PS 1546’ lettuce seed. In another embodiment, the present invention is directed to a Lactuca sativa plant and parts isolated therefrom having all the physiological and morphological characteristics of a Lactuca sativa plant produced by growing ‘PS 1546’ lettuce seed having ATCC Accession Number X4. In still another embodiment, the present invention is directed to an F₁ hybrid Lactuca sativa lettuce seed, plants grown from the seed, and a head isolated therefrom having ‘PS 1546’ as a parent, where ‘PS 1546’ is grown from ‘PS 1546’ lettuce seed having ATCC Accession Number X4.

Lettuce plant parts include lettuce heads, lettuce leaves, parts of lettuce leaves, pollen, ovules, flowers, and the like. In another embodiment, the present invention is further directed to lettuce heads, lettuce leaves, parts of lettuce leaves, flowers, pollen, and ovules isolated from ‘PS 1546’ lettuce plants. In another embodiment, the present invention is further directed to tissue or cell culture of ‘PS 1546’ lettuce plants, and to lettuce plants regenerated from the tissue or cell culture, where the plant has all of the morphological and physiological characteristics of ‘PS 1546’ lettuce plants. In another embodiment, the present invention is further directed to tissue cell culture produced from protoplasts or cells of a lettuce plant, wherein the lettuce plant is produced by growing ‘PS 1546’ lettuce seed having ATCC Accession Number X4.

The present invention is further directed to a method of selecting lettuce plants by: a) growing more than one ‘PS 1546’ lettuce plant, where the plants are grown from lettuce seed having ATCC Accession Number X4; and b) selecting a plant from step a). The present invention is further directed to lettuce plants and seeds produced therefrom, where the lettuce plants and seeds are isolated by the selection method of the invention.

In another embodiment, the present invention is further directed to a method of breeding lettuce plants by crossing a lettuce plant with a plant grown from ‘PS 1546’ lettuce seed having ATCC Accession Number X4. In still another embodiment, the present invention is further directed to lettuce plants, lettuce parts from the lettuce plants (e.g., lettuce heads), and seeds produced therefrom where the lettuce plant is isolated by the breeding method of the invention.

In one embodiment, the present invention is directed to lettuce, Lactuca sativa, seed designated as ‘PS 1902’ having ATCC Accession Number X11. In one embodiment, the present invention is directed to a Lactuca sativa lettuce plant and parts isolated therefrom produced by growing ‘PS 1902’ lettuce seed. In another embodiment, the present invention is directed to a Lactuca sativa plant and parts isolated therefrom having all the physiological and morphological characteristics of a Lactuca sativa plant produced by growing ‘PS 1902’ lettuce seed having ATCC Accession Number X11. In still another embodiment, the present invention is directed to an F₁ hybrid Lactuca sativa lettuce seed, plants grown from the seed, and a head isolated therefrom having ‘PS 1902’ as a parent, where ‘PS 1902’ is grown from ‘PS 1902’ lettuce seed having ATCC Accession Number X11.

Lettuce plant parts include lettuce heads, lettuce leaves, parts of lettuce leaves, pollen, ovules, flowers, and the like. In another embodiment, the present invention is further directed to lettuce heads, lettuce leaves, parts of lettuce leaves, flowers, pollen, and ovules isolated from ‘PS 1902’ lettuce plants. In another embodiment, the present invention is further directed to tissue or cell culture of ‘PS 1902’ lettuce plants, and to lettuce plants regenerated from the tissue or cell culture, where the plant has all of the morphological and physiological characteristics of ‘PS 1902’ lettuce plants. In another embodiment, the present invention is further directed to tissue cell culture produced from protoplasts or cells of a lettuce plant, wherein the lettuce plant is produced by growing ‘PS 1902’ lettuce seed having ATCC Accession Number X11.

The present invention is further directed to a method of selecting lettuce plants by: a) growing more than one ‘PS 1902’ lettuce plant, where the plants are grown from lettuce seed having ATCC Accession Number X11; and b) selecting a plant from step a). The present invention is further directed to lettuce plants and seeds produced therefrom, where the lettuce plants and seeds are isolated by the selection method of the invention.

In another embodiment, the present invention is further directed to a method of breeding lettuce plants by crossing a lettuce plant with a plant grown from ‘PS 1902’ lettuce seed having ATCC Accession Number X11. In still another embodiment, the present invention is further directed to lettuce plants, lettuce parts from the lettuce plants (e.g., lettuce heads), and seeds produced therefrom where the lettuce plant is isolated by the breeding method of the invention.

In another embodiment, the present invention relates to a method of making Lactuca sativa seeds, including crossing the lettuce plant of any of the preceding embodiments with another lettuce plant and harvesting seed therefrom. In some embodiments, the method includes planting the seed to produce F1 progeny. In some embodiments, the method further includes (a) selecting one or more of the F1 progeny for plants that have a desired trait to produce selected progeny plants; (b) crossing the selected progeny plants with the lettuce plant of any of the preceding embodiments to produce backcross progeny plants; (c) selecting for backcross progeny plants that have the desired trait and all of the physiological and morphological characteristics of the lettuce plant of any of the preceding embodiments when grown in the same environmental conditions to produce selected backcross progeny plants; and (d) repeating steps (b) and (c) three or more times in succession to produce selected fourth or higher backcross progeny plants that comprise the desired trait and all of the physiological and morphological characteristics of the lettuce plant of any of the preceding embodiments when grown in the same environmental conditions.

In another aspect, the present invention relates to a method of producing a seed of a lettuce plant derived from the lettuce plant of any of the preceding embodiments, including crossing a Lactuca sativa plant of any of the preceding embodiments with a second lettuce plant, whereby seed of a derived lettuce plant forms. In another embodiment, the present invention relates to a derived lettuce plant produced by growing the seed of any of the preceding embodiments. In some embodiments, the method further includes (a) crossing a plant grown from Lactuca sativa seed derived from the lettuce plant of any of the preceding embodiments with itself or with a second lettuce plant to yield additional seed of a derived lettuce plant; (b) growing the additional derived lettuce plants; and (c) repeating the crossing and growing of steps (a) and (b) for an additional 3-10 generations to generate further lettuce plants derived from the lettuce plant of any of the preceding embodiments.

In another embodiment, the present invention relates to a method of producing a lettuce leaf, heart, or head from the plant of any of the preceding embodiments, said method including growing the plant of any of the preceding embodiments until it produces at least one leaf, heart, or head; and harvesting the leaf, heart, or head.

### BRIEF DESCRIPTION OF THE DRAWINGS

The patent or application file contains at least one drawing executed in color. Copies of this patent or patent application publication with color drawings will be provided by the office upon request and payment of the necessary fee.

FIGS. 1A-1S show comparisons of lettuce variety ‘PS 1540’ (‘Jackpot’) with lettuce varieties ‘PS 1523’ (‘Latitude’, patent pending) and ‘PS 1272’ (‘Newcastle’, U.S. Pat. No. 11,944,054). FIG. 1A shows a juvenile leaf of lettuce variety ‘PS 1540’. FIG. 1B shows a juvenile leaf of lettuce variety ‘Latitude’. FIG. 1C shows a juvenile leaf of lettuce variety ‘Newcastle’. FIG. 1D shows bolting plants of lettuce variety ‘PS 1540’. FIG. 1E shows bolting plants of lettuce variety ‘Latitude’. FIG. 1F shows bolting plants of lettuce variety ‘Newcastle’. FIG. 1G shows seedlings of lettuce variety ‘PS 1540’. FIG. 1H shows seedlings of lettuce variety ‘Latitude’. FIG. 1I shows seedlings of lettuce variety ‘Newcastle’. FIG. 1J shows a top view of a plant of lettuce variety ‘PS 1540’ (left) and a plant of lettuce variety ‘Latitude’ (right). FIG. 1K shows a top view of a plant of lettuce variety ‘PS 1540’ (left) and a plant of lettuce variety ‘Newcastle’ (right). FIG. 1L shows a bottom view of a head of lettuce variety ‘PS 1540’ (left) and lettuce variety ‘Latitude’ (right). FIG. 1M shows a bottom view of a head of lettuce variety ‘PS 1540’ (left) and lettuce variety ‘Newcastle’ (right). FIG. 1N shows a side view of a head of lettuce variety ‘PS 1540’ (left) and lettuce variety ‘Latitude’ (right). FIG. 1O shows a side view of a head of lettuce variety ‘PS 1540’ (left) and lettuce variety ‘Newcastle’ (right). FIG. 1P shows a cross-sectional view of a head of lettuce variety ‘PS 1540’ (left) and lettuce variety ‘Latitude’ (right). FIG. 1Q shows a cross-sectional view of a head of lettuce variety ‘PS 1540’ (left) and lettuce variety ‘Newcastle’ (right). FIG. 1R shows a mature leaf of lettuce variety ‘PS 1540’ (left) and a mature leaf of lettuce variety ‘Latitude’ (right). FIG. 1S shows a mature leaf of lettuce variety ‘PS 1540’ (left) and a mature leaf of lettuce variety ‘Newcastle’ (right).

FIGS. 2A-2S show comparisons of lettuce variety ‘PS 1541’ (‘Keystone’) with lettuce varieties ‘PS 1523’ (‘Latitude’, patent pending) and ‘PS 1272’ (‘Newcastle’, U.S. Pat. No. 11,944,054). FIG. 2A shows a juvenile leaf of lettuce variety ‘PS 1541’. FIG. 2B shows a juvenile leaf of lettuce variety ‘Latitude’. FIG. 2C shows a juvenile leaf of lettuce variety ‘Newcastle’. FIG. 2D shows bolting plants of lettuce variety ‘PS 1541’. FIG. 2E shows bolting plants of lettuce variety ‘Latitude’. FIG. 2F shows bolting plants of lettuce variety ‘Newcastle’. FIG. 2G shows seedlings of lettuce variety ‘PS 1541’. FIG. 2H shows seedlings of lettuce variety ‘Latitude’. FIG. 2I shows seedlings of lettuce variety ‘Newcastle’. FIG. 2J shows a top view of a plant of lettuce variety ‘PS 1541’ (left) and a plant of lettuce variety ‘Latitude’ (right). FIG. 2K shows a top view of a plant of lettuce variety ‘PS 1541’ (left) and a plant of lettuce variety ‘Newcastle’ (right). FIG. 2L shows a bottom view of a head of lettuce variety ‘PS 1541’ (left) and lettuce variety ‘Latitude’ (right). FIG. 2M shows a bottom view of a head of lettuce variety ‘PS 1541’ (left) and lettuce variety ‘Newcastle’ (right). FIG. 2N shows a side view of a head of lettuce variety ‘PS 1541’ (left) and lettuce variety ‘Latitude’ (right). FIG. 2O shows a side view of a head of lettuce variety ‘PS 1541’ (left) and lettuce variety ‘Newcastle’ (right). FIG. 2P shows a cross-sectional view of a head of lettuce variety ‘PS 1541’ (left) and lettuce variety ‘Latitude’ (right). FIG. 2Q shows a cross-sectional view of a head of lettuce variety ‘PS 1541’ (left) and lettuce variety ‘Newcastle’ (right). FIG. 2R shows a mature leaf of lettuce variety ‘PS 1541’ (left) and a mature leaf of lettuce variety ‘Latitude’ (right). FIG. 2S shows a mature leaf of lettuce variety ‘PS 1541’ (left) and a mature leaf of lettuce variety ‘Newcastle’ (right).

FIGS. 3A-3S show comparisons of lettuce variety ‘PS 1546’ with lettuce varieties ‘Salute’ (U.S. Pat. No. 8,835,719) and ‘Meridian’ (U.S. Pat. No. 9,706,743). FIG. 3A shows a juvenile leaf of lettuce variety ‘PS 1546’. FIG. 3B shows a juvenile leaf of lettuce variety ‘Salute’. FIG. 3C shows a juvenile leaf of lettuce variety ‘Meridian’. FIG. 3D shows bolting plants of lettuce variety ‘PS 1546’. FIG. 3E shows bolting plants of lettuce variety ‘Salute’. FIG. 3F shows bolting plants of lettuce variety ‘Meridian’. FIG. 3G shows seedlings of lettuce variety ‘PS 1546’. FIG. 3H shows seedlings of lettuce variety ‘Salute’. FIG. 3I shows seedlings of lettuce variety ‘Meridian’. FIG. 3J shows a top view of a plant of lettuce variety ‘PS 1546’ (left) and a plant of lettuce variety ‘Salute’ (right). FIG. 3K shows a top view of a plant of lettuce variety ‘PS 1546’ (left) and a plant of lettuce variety ‘Meridian’ (right). FIG. 3L shows a bottom view of a head of lettuce variety ‘PS 1546’ (left) and lettuce variety ‘Salute’ (right). FIG. 3M shows a bottom view of a head of lettuce variety ‘PS 1546’ (left) and lettuce variety ‘Meridian’ (right). FIG. 3N shows a side view of a head of lettuce variety ‘PS 1546’ (left) and lettuce variety ‘Salute’ (right). FIG. 3O shows a side view of a head of lettuce variety ‘PS 1546’ (left) and lettuce variety ‘Meridian’ (right). FIG. 3P shows a cross-sectional view of a head of lettuce variety ‘PS 1546’ (left) and lettuce variety ‘Salute’ (right). FIG. 3Q shows a cross-sectional view of a head of lettuce variety ‘PS 1546’ (left) and lettuce variety ‘Meridian’ (right). FIG. 3R shows a mature leaf of lettuce variety ‘PS 1546’ (left) and a mature leaf of lettuce variety ‘Salute’ (right). FIG. 3S shows a mature leaf of lettuce variety ‘PS 1546’ (left) and a mature leaf of lettuce variety ‘Meridian’ (right).

FIGS. 4A-4T show comparisons of lettuce variety ‘PS 1902’ with lettuce varieties ‘Big Star’ (U.S. Plant Variety Protection Certificate 200200013) and ‘Bergam's Green’ (U.S. Plant Variety Protection Certificate 200500262). FIG. 4A shows a juvenile leaf of lettuce variety ‘PS 1902’. FIG. 4B shows a juvenile leaf of lettuce variety ‘Big Star’. FIG. 4C shows a juvenile leaf of lettuce variety ‘Bergam's Green’. FIG. 4D shows bolting plants of lettuce variety ‘PS 1902’. FIG. 4E shows bolting plants of lettuce variety ‘Big Star’. FIG. 4F shows bolting plants of lettuce variety ‘Bergam's Green’. FIG. 4G shows seedlings of lettuce variety ‘PS 1902’. FIG. 4H shows seedlings of lettuce variety ‘Big Star’. FIG. 4I shows seedlings of lettuce variety ‘Bergam's Green’. FIG. 4J shows a top view of a plant of lettuce variety ‘PS 1902’. FIG. 4K shows a top view of a plant of lettuce variety ‘Big Star’. FIG. 4L shows a top view of a plant of lettuce variety ‘Bergam's Green’. FIG. 4M shows a bottom view of a head of lettuce variety ‘PS 1902’ (left) and lettuce variety ‘Big Star’ (right). FIG. 4N shows a bottom view of a head of lettuce variety ‘PS 1902’ (left) and lettuce variety ‘Bergam's Green’ (right). FIG. 4O shows a side view of a head of lettuce variety ‘PS 1902’ (left) and lettuce variety ‘Big Star’ (right). FIG. 4P shows a side view of a head of lettuce variety ‘PS 1902’ (left) and lettuce variety ‘Bergam's Green’ (right). FIG. 4Q shows a cross-sectional view of a head of lettuce variety ‘PS 1902’ (left) and lettuce variety ‘Big Star’ (right). FIG. 4R shows a cross-sectional view of a head of lettuce variety ‘PS 1902’ (left) and lettuce variety ‘Bergam's Green’ (right). FIG. 4S shows a mature leaf of lettuce variety ‘PS 1902’ (left) and a mature leaf of lettuce variety ‘Big Star’ (right). FIG. 4T shows a mature leaf of lettuce variety ‘PS 1902’ (left) and a mature leaf of lettuce variety ‘Bergam's Green’ (right).

### DETAILED DESCRIPTION

### Definitions

In order to more clearly understand the invention, the following definitions are provided:

Core Length: Core length is the length of the internal lettuce stem. Core length is measured from the base of the cut head to the tip of the core.

Core Diameter: Core diameter is the diameter of the lettuce stem at the base of the cut head.

Fusarium Wilt: Fusarium wilt of lettuce is a disease caused by the fungus Fusarium oxysporum f. sp. lactucae that causes infected seedlings to wilt, and turn red or brown in color in inner tissues, and causes leaves of infected older plants to turn yellow and develop tip burn.

Heart: Heart is the portion in the center of romaine type lettuces where the leaf tips curve inward to cover the growing point. Cut and trimmed hearts of romaine type lettuces can be obtained by removing the frame leaves and cutting the stem off just below the base of the outermost heart leaf.

Heart Length: Heart length is the length of the vertically sliced romaine type lettuce plant as measured from the base of the cut stem to the top leaf margin of the longest outermost leaf that encloses the green leaf heart.

Head Diameter: Head diameter is the diameter of the vertically sliced iceberg type lettuce plant head at its widest horizontal point, perpendicular to the stem.

Head Length: Head length is the diameter of the vertically sliced iceberg type lettuce plant head as measured from the base of the cut stem to the cap leaf.

Head Weight: Head weight is the weight of the marketable iceberg type lettuce plant, cut and trimmed to market specifications.

Impatiens Necrotic Spot Virus (INSV): A disease that can cause dead spots, stunted plant growth, and yellowing leaves in outer lettuce leaves. Infected lettuce may have necrotic and chlorotic inner leaves, and the base of infected lettuce plants' ribs exhibit necrosis and lesions. An early infection may lead to stunted lettuce plant growth.

Lettuce Mosaic Virus: A disease that can cause a stunted, deformed, or mottled pattern in young lettuce and yellow, twisted, and deformed leaves in older lettuce.

Maturity Date: Maturity refers to the stage when the plants are of full size or optimum weight, in marketable form or shape to be of commercial or economic value.

Nasonovia ribisnigri: A lettuce aphid that colonizes the innermost leaves of the lettuce plant, contaminating areas that cannot be treated easily with insecticides.

Plant Diameter: The plant diameter is a measurement across the top of the lettuce plant at its widest point. The measurement of frame diameter is taken from the outer most leaf tip horizontally to the outer most leaf tip.

Tip burn: Means a browning of the edges or tips of lettuce leaves that is a physiological response to a lack of calcium.

Tomato Bushy Stunt: Lettuce dieback was first observed in California in the mid-1980s, and reports of the disease have increased over the last 10 years. The disease has occurred in commercial fields of some leaf lettuce cultivars; however, symptoms have never been observed on any modern crisphead (iceberg) cultivars. Complete crop losses have occurred in fields of green leaf lettuce, and no commercial green leaf cultivar has been shown to be resistant to the disease. Lettuce dieback is caused by several related tombusviruses including tomato bushy stunt virus (TBSV) and lettuce necrotic stunt virus (LNSV) (Liu et al., 1999; Obermeier et al., 2001). These are soil-borne, highly stable, and mechanically transmitted, and have no known vector. The conditions affecting symptom development remain poorly understood. The disease is frequently observed in low-lying areas of fields with a prior history of flooding, suggesting that the virus may be carried in river water and/or that disease symptoms may be associated with increased root stresses such as those presented by excess moisture. No effective cultural or chemical control methods have yet been identified.

Resistance to Tomato Busy Stunt refers to a level of resistance in a lettuce variety as measured by visual symptoms. Resistance is deemed present when symptoms are not present in at least 95% of a lettuce variety when exposed to tomato bushy stunt virus (TBSV). Verticillium Wilt: Verticillium Wilt of lettuce is a disease caused by the fungus Verticillium dahlia that can cause the basal leaves that cover the outer part of the lettuce head to wilt and then collapse, leading to premature plant death and an unharvestable head.

Taking into account these definitions, the present invention is directed to seeds of the lettuce varieties ‘PS 1540’, ‘PS 1541’, ‘PS 1546’, and ‘PS 1902’, plants produced by growing ‘PS 1540’, ‘PS 1541’, ‘PS 1546’, and/or ‘PS 1902’ lettuce seeds, heads isolated or harvested from the plants, one or more plants selected from a collection of ‘PS 1540’, ‘PS 1541’, ‘PS 1546’, and/or ‘PS 1902’ plants and seeds derived or produced therefrom; plants produced by crossing a lettuce plant with a ‘PS 1540’, ‘PS 1541’, ‘PS 1546’, and/or ‘PS 1902’ lettuce plant and seeds derived or produced therefrom.

### Objective Description of the Variety ‘PS 1540’

‘PS 1540’ is an inbred iceberg lettuce variety. This variety is distinct and unique to all other iceberg lettuce varieties due to its Fusarium resistance and Verticillium resistance. Moreover, ‘PS 1540’ is designed to be able to be grown in a grow box or as processing lettuce in large bed configurations. ‘PS 1540’ has strong heading, short core, high tip burn resistance, and Verticillium resistance. When compared to reference varieties, ‘PS 1540’ has larger frame length and smaller core diameter. ‘PS 1540’ is adapted to growing in regions such as the Southwest and West Coast regions of the United States, such as California and the Arizona desert. In particular, ‘PS 1540’ is suitable for growing in the spring for areas such as Yuma, Arizona, USA, and in the summer for areas such as Salinas, California, USA. Lettuce variety ‘PS 1540’ is the result of numerous generations of plant selections chosen for its Fusarium resistance, and Verticillium resistance.

The variety has shown uniformity and stability for the traits, within the limits of environmental influence for the traits. It has been self-pollinated a sufficient number of generations with careful attention to uniformity of plant type. The line has been increased with continued observation for uniformity. No variant traits have been observed or are expected in variety ‘PS 1540’.
Lettuce Variety ‘PS 1540’ has the Following Morphologic and Other Characteristics:
Plant type: Crisp (i.e., iceberg)
Seed:
Color: White (silver gray)
Light dormancy: Light not required
Heat dormancy: Susceptible
Cotyledon to Fourth Leaf Stage:
Shape of cotyledons: Spatulate
Cotyledon length: 11.9 mm
Cotyledon width: 7.1 mm
Cotyledon index (length/width×10): 16.7
Shape of fourth leaf: Elongated
Fourth leaf length: 23.5 mm
Fourth leaf width: 56.3 mm
Fourth leaf index (length/width ×10): 4.2
Apical margin: Finely dentate
Basal margin: Moderately dentate
Green color: Dark green
Anthocyanin distribution: Absent
Cupping: Uncupped
Reflexing: Apical margin
Mature Leaves:
Margin:
Incision depth (deepest penetration of the margin): Moderate
Indentation (finest divisions of the margin): Crenate
Undulation of apical margin: Moderate
Color (Munsell): 5GY 5/4 (Dark green)
Anthocyanin distribution: Absent
Glossiness: Moderate
Blistering: Absent/slight
Thickness: Intermediate
Trichomes: Absent (smooth)
Plant:
Weight: 581.1 g
Spread of frame leaves: 45.9 cm
Head diameter (market trimmed with single cap leaf): 12.9 cm
Head Shape: Slightly flattened
Head size class: Medium
Head firmness: Firm
Butt:
Shape: Rounded
Midrib: Flattened
Plant Core:
Diameter at base of head: 27.7 mm
Ratio of head diameter/core diameter: 4.7
Core height from base to head to apex: 31.4 mm
Bolting:
Number of days from first water to seed stalk emergence under summer conditions: 67
Bolting class: Medium
Mature seed stalk height: 95.7 cm
Mature seed stalk spread: 27.3 cm
Bolter leaves: Curved
Margin: Entire
Bolter habit:
Terminal inflorescence: Present
Lateral shoots: Present
Basal side shoots: Absent
Disease/Pest Resistance:
Downy Mildew (Bremia lactucae) (B1): Intermediate Resistant
Verticillium Race 1: Highly Resistant
Fusarium Race 1: Intermediate Resistant
Nasonovia aphid (Nasonovia ribisnigri) biotype 0 (Nr: 0): Susceptible
Stress Resistance:
Tipburn: High tip burn resistance

### Comparisons to Commercial Lettuce Variety

Table 1 below compares characteristics of lettuce variety ‘PS 1540’ with the lettuce varieties ‘Latitude’ (patent pending) and ‘Newcastle’ (U.S. Pat. No. 11,944,054). Column 1 lists the characteristics, column 2 shows the characteristics for lettuce variety ‘PS 1540’, column 3 shows the characteristics for lettuce variety ‘Latitude’, and column 4 shows the characteristics for lettuce variety ‘Newcastle’.

TABLE 1

| | | | |
|---|---|---|---|
| Characteristic | ‘PS 1540’ | ‘Latitude’ | ‘Newcastle’ |

| | | | |
|---|---|---|---|
| Seed Color | White | Black | Black |
| | (Silver gray) | | |
| Verticillium | Highly | Resistant | Highly |
| Resistance | Resistant | | Resistant |
| Fusarium | Intermediate | Susceptible | — |
| Resistance | Resistant | | |

| | | | | | | |
|---|---|---|---|---|---|---|
| Frame Length | 45.9 | cm | 44.9 | cm | 45.8 | cm |
| Weight | 581.1 | g | 650.5 | g | 582.8 | g |
| Head Diameter | 12.9 | mm | 13.3 | mm | 13.1 | mm |
| Core Diameter | 27.7 | mm | 29.3 | mm | 30.2 | mm |
| Core Length | 31.4 | mm | 38.9 | mm | 34.6 | mm |

| | | | |
|---|---|---|---|
| Color | 5GY 5/4 | 5GY 5/6 | 2.5 GY 5/6 |
| (Munsell) | | | |

Tables 2A-2C below show results of a first trial that compares the head weight, head diameter, core length, core diameter, and frame diameter of 20 plants of the lettuce variety ‘PS 1540’ (Table 2A) with those of 20 plants of lettuce variety ‘Latitude’ (Table 2B) and 20 plants of lettuce variety ‘Newcastle’ (Table 2C). The head weights shown are total head weights.

TABLE 2A

| | | Head | Core | Core | Frame |
|---|---|---|---|---|---|
| | Head Wt. | Diameter | Length | Diameter | Diameter |
| ‘PS 1540’ | (g) | (cm) | (mm) | (mm) | (cm) |
| Max | 845 | 14.5 | 48 | 36 | 52.6 |
| Min | 411 | 10.9 | 31 | 22 | 46.1 |
| Average | 644.4 | 13.14 | 39.3 | 30.9 | 49.7 |
| Std. Dev. | 109.5 | 0.7528 | 4.791 | 3.339 | 1.911 |

TABLE 2B

| | | Head | Core | Core | Frame |
|---|---|---|---|---|---|
| | Head Wt. | Diameter | Length | Diameter | Diameter |
| ‘Latitude’ | (g) | (cm) | (mm) | (mm) | (cm) |
| Max | 975 | 15.6 | 59 | 38 | 50.7 |
| Min | 558 | 12.1 | 34 | 30 | 44.1 |
| Average | 738.1 | 13.8 | 46.0 | 34.5 | 47.5 |
| Std. Dev. | 118.2 | 1.031 | 5.885 | 2.260 | 1.921 |

TABLE 2C

| | | Head | Core | Core | Frame |
|---|---|---|---|---|---|
| | Head Wt. | Diameter | Length | Diameter | Diameter |
| ‘Newcastle’ | (g) | (cm) | (mm) | (mm) | (cm) |
| Max | 952 | 16.6 | 44 | 35 | 49.8 |
| Min | 504 | 11.2 | 33 | 29 | 43.2 |
| Average | 631.4 | 14.0 | 37.0 | 32.3 | 46.8 |
| Std. Dev. | 102.4 | 1.297 | 3.026 | 1.832 | 1.744 |

Tables 3A-3C below show results of a second trial that compares the head weight, head diameter, core length, core diameter, and frame diameter of 20 plants of the lettuce variety ‘PS 1540’ (Table 3A) with those of 20 plants of lettuce variety ‘Latitude’ (Table 3B) and 20 plants of lettuce variety ‘Newcastle’ (Table 3C). The head weights shown are total head weights.

TABLE 3A

| | | Head | Core | Core | Frame |
|---|---|---|---|---|---|
| | Head Wt. | Diameter | Length | Diameter | Diameter |
| ‘PS 1540’ | (g) | (cm) | (mm) | (mm) | (cm) |
| Max | 873 | 14.5 | 38 | 30 | 49.6 |
| Min | 481 | 11.8 | 21 | 23 | 38.4 |
| Average | 719.1 | 13.0 | 27.9 | 26.4 | 43.8 |
| Std. Dev. | 99.12 | 0.6837 | 4.254 | 2.300 | 2.554 |

TABLE 3B

| | | Head | Core | Core | Frame |
|---|---|---|---|---|---|
| | Head Wt. | Diameter | Length | Diameter | Diameter |
| ‘Latitude’ | (g) | (cm) | (mm) | (mm) | (cm) |
| Max | 1060 | 15.5 | 47 | 35 | 45.6 |
| Min | 554 | 11.6 | 23 | 20 | 38.4 |
| Average | 837.4 | 13.8 | 32.3 | 26 | 42.1 |
| Std. Dev. | 150.5 | 0.9550 | 5.555 | 3.934 | 1.9632 |

TABLE 3C

| | | Head | Core | Core | Frame |
|---|---|---|---|---|---|
| | Head Wt. | Diameter | Length | Diameter | Diameter |
| ‘Newcastle’ | (g) | (cm) | (mm) | (mm) | (cm) |
| Max | 954 | 14.3 | 34 | 38 | 48.4 |
| Min | 511 | 8.6 | 18 | 22 | 39.4 |
| Average | 682.9 | 12.5 | 24.8 | 27.8 | 42.8 |
| Std. Dev. | 111.8 | 1.384 | 3.492 | 3.365 | 2.146 |

Tables 4A-4C below show results of a third trial that compares the head weight, head diameter, core length, core diameter, and frame diameter of 20 plants of the lettuce variety ‘PS 1540’ (Table 4A) with those of 20 plants of lettuce variety ‘Latitude’ (Table 4B) and 20 plants of lettuce variety ‘Newcastle’ (Table 4C). The head weights shown are total head weights.

TABLE 4A

| | | Head | Core | Core | Frame |
|---|---|---|---|---|---|
| | Head Wt. | Diameter | Length | Diameter | Diameter |
| ‘PS 1540’ | (g) | (cm) | (mm) | (mm) | (cm) |
| Max | 688 | 13.5 | 38 | 31 | 49.1 |
| Min | 407 | 10.8 | 25 | 24 | 42.1 |
| Average | 547.3 | 12.2 | 30.9 | 27.3 | 46.0 |
| Std. Dev. | 80.78 | 0.6112 | 3.754 | 2.055 | 2.119 |

TABLE 4B

| | | Head | Core | Core | Frame |
|---|---|---|---|---|---|
| | Head Wt. | Diameter | Length | Diameter | Diameter |
| ‘Latitude’ | (g) | (cm) | (mm) | (mm) | (cm) |
| Max | 759 | 14.7 | 39 | 34 | 50.3 |
| Min | 433 | 10.2 | 27 | 26 | 44.3 |
| Average | 598.8 | 12.6 | 33.9 | 30.6 | 46.9 |
| Std. Dev. | 101.2 | 1.327 | 3.249 | 2.139 | 1.583 |

TABLE 4C

| | | Head | Core | Core | Frame |
|---|---|---|---|---|---|
| | Head Wt. | Diameter | Length | Diameter | Diameter |
| ‘Newcastle’ | (g) | (cm) | (mm) | (mm) | (cm) |
| Max | 739 | 14.9 | 36 | 36 | 52.1 |
| Min | 352 | 10.2 | 19 | 25 | 44.3 |
| Average | 535.4 | 12.4 | 27.3 | 30.8 | 48.4 |
| Std. Dev. | 89.41 | 1.077 | 4.354 | 2.845 | 2.375 |

Tables 5A-5C below show results of a fourth trial that compares the head weight, head diameter, core length, core diameter, and frame diameter of 20 plants of the lettuce variety ‘PS 1540’ (Table 5A) with those of 20 plants of lettuce variety ‘Latitude’ (Table 5B) and 20 plants of lettuce variety ‘Newcastle’ (Table 5C). The head weights shown are total head weights.

TABLE 5A

| | | Head | Core | Core | Frame |
|---|---|---|---|---|---|
| | Head Wt. | Diameter | Length | Diameter | Diameter |
| ‘PS 1540’ | (g) | (cm) | (mm) | (mm) | (cm) |
| Max | 578.8 | 14.3 | 33 | 32 | 50.3 |
| Min | 236.4 | 11.7 | 22 | 24 | 39.8 |
| Average | 413.6 | 13.1 | 27.4 | 26.2 | 44.4 |
| Std. Dev. | 88.02 | 0.6562 | 3.297 | 1.755 | 2.830 |

TABLE 5B

| | | Head | Core | Core | Frame |
|---|---|---|---|---|---|
| | Head Wt. | Diameter | Length | Diameter | Diameter |
| ‘Latitude’ | (g) | (cm) | (mm) | (mm) | (cm) |
| Max | 623.4 | 14.5 | 57 | 29 | 49.7 |
| Min | 261.5 | 11.1 | 31 | 22 | 37.6 |
| Average | 427.7 | 13.0 | 43.6 | 26.1 | 43.0 |
| Std. Dev. | 86.91 | 1.071 | 5.790 | 1.791 | 3.766 |

TABLE 5C

| | | Head | Core | Core | Frame |
|---|---|---|---|---|---|
| | Head Wt. | Diameter | Length | Diameter | Diameter |
| ‘Newcastle’ | (g) | (cm) | (mm) | (mm) | (cm) |
| Max | 692.6 | 15.8 | 61 | 33 | 53.4 |
| Min | 315.7 | 11.8 | 35 | 26 | 41.2 |
| Average | 481.5 | 13.6 | 49.3 | 29.9 | 45.4 |
| Std. Dev. | 109.9 | 0.9544 | 8.117 | 2.100 | 3.013 |

Tables 6A-6C below compares the fourth leaf width, fourth leaf length, cotyledon leaf width, cotyledon leaf length, stalk length, and frame of 20 plants of the lettuce variety ‘PS 1540’ (Table 6A) with those of 20 plants of lettuce variety ‘Latitude’ (Table 6B) and 20 plants of lettuce variety ‘Newcastle’ (Table 6C). The head weights shown are total head weights.

TABLE 6A

| | Fourth | Fourth | Cot. | | | |
|---|---|---|---|---|---|---|
| | leaf | leaf | leaf | Cot. leaf | | |
| | width | length | width | length | Stalk | Frame |
| ‘PS 1540’ | (mm) | (mm) | (mm) | (mm) | (cm) | (cm) |
| Max | 69 | 29 | 8 | 14 | 102.4 | 32.8 |
| Min | 42 | 20 | 6 | 9 | 88.5 | 23.8 |
| Average | 56.3 | 23.5 | 7.1 | 11.9 | 95.7 | 27.3 |
| Std. Dev. | 6.727 | 2.114 | 0.7881 | 1.309 | 3.908 | 2.276 |

TABLE 6B

| | Fourth | Fourth | Cot. | | | |
|---|---|---|---|---|---|---|
| | leaf | leaf | leaf | Cot. leaf | | |
| | width | length | width | length | Stalk | Frame |
| ‘Latitude’ | (mm) | (mm) | (mm) | (mm) | (cm) | (cm) |
| Max | 71 | 28 | 9 | 14 | 108.4 | 33.2 |
| Min | 50 | 22 | 6 | 9 | 86.5 | 23.2 |
| Average | 60.6 | 24.2 | 7.5 | 11.7 | 98.4 | 28.7 |
| Std. Dev. | 5.256 | 1.631 | 0.7592 | 1.565 | 6.134 | 2.097 |

TABLE 6C

| | Fourth | Fourth | Cot. | | | |
|---|---|---|---|---|---|---|
| | leaf | leaf | leaf | Cot. leaf | | |
| | width | length | width | length | Stalk | Frame |
| ‘Newcastle’ | (mm) | (mm) | (mm) | (mm) | (cm) | (cm) |
| Max | 57 | 23 | 8 | 15 | 104.2 | 29.4 |
| Min | 43 | 20 | 5 | 9 | 85.7 | 22.8 |
| Average | 51.4 | 21.2 | 7.2 | 11.7 | 93.5 | 25.9 |
| Std. Dev. | 3.858 | 1.056 | 0.8127 | 1.525 | 5.235 | 1.781 |

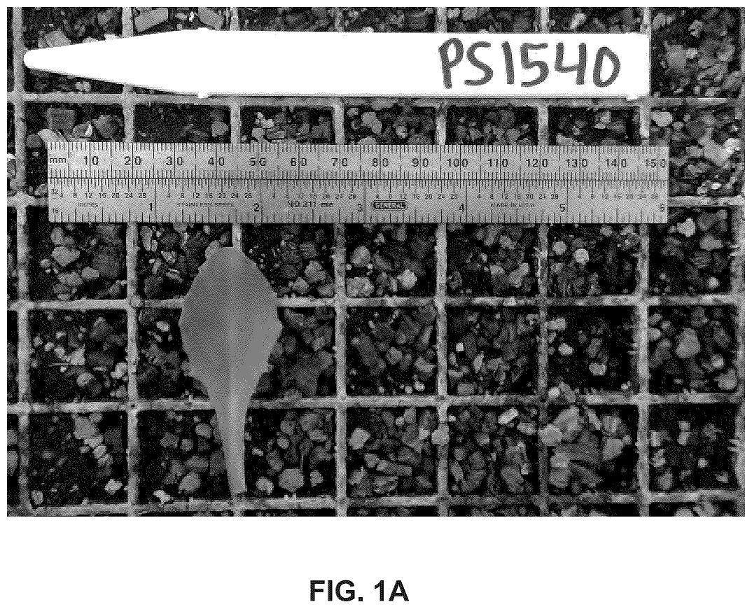

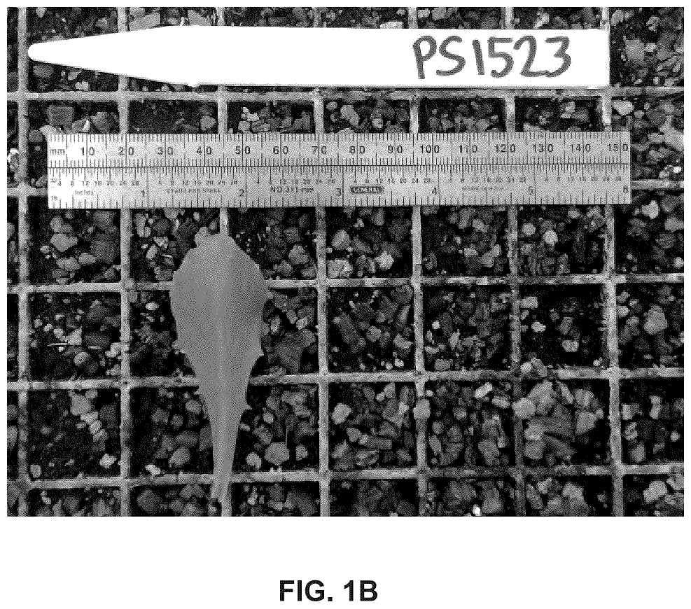

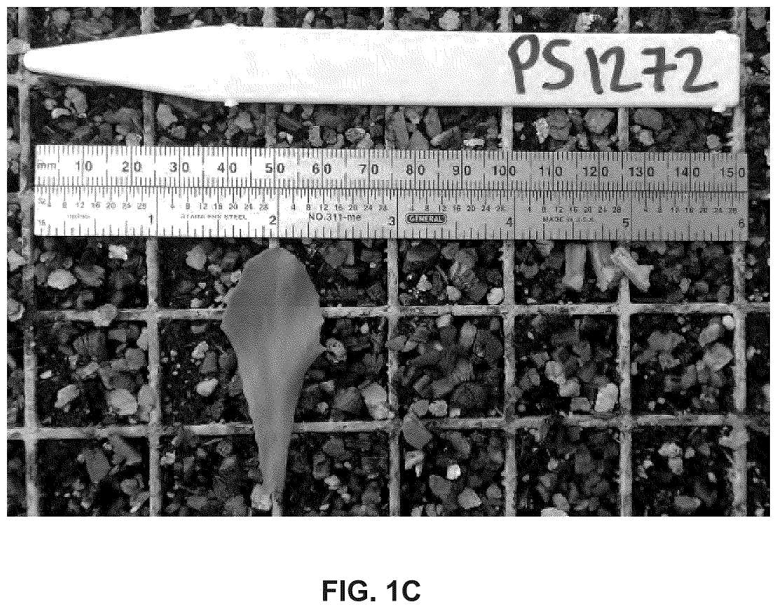

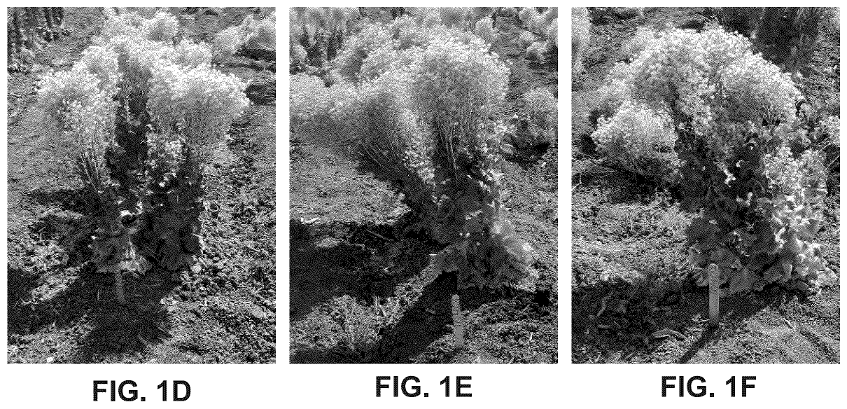

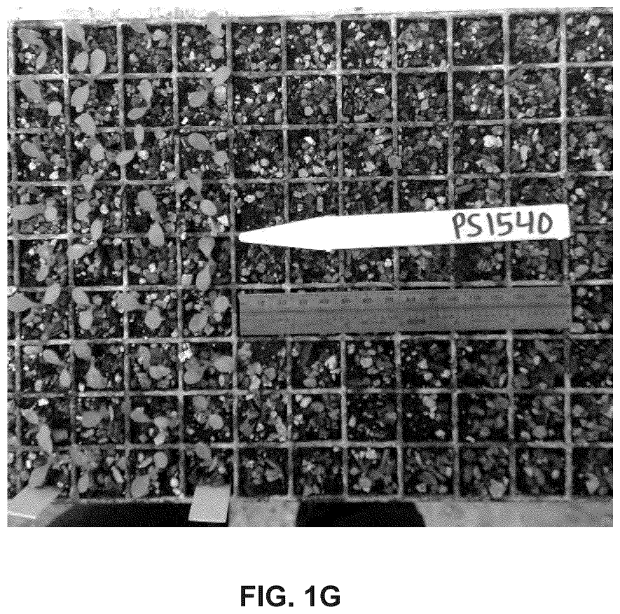

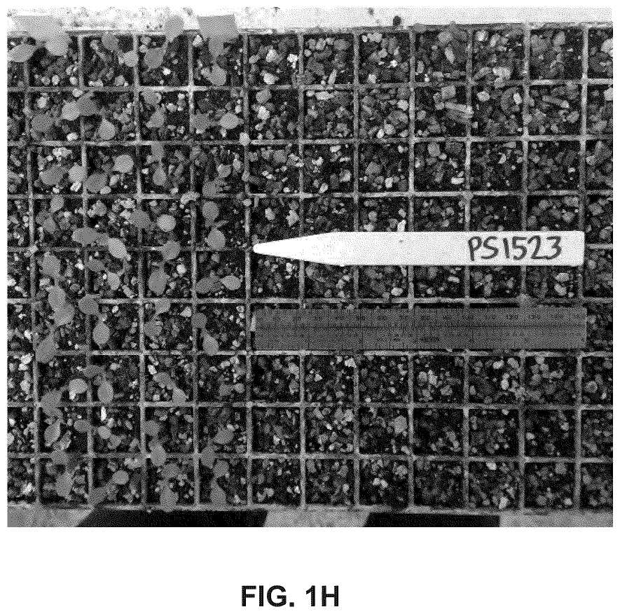

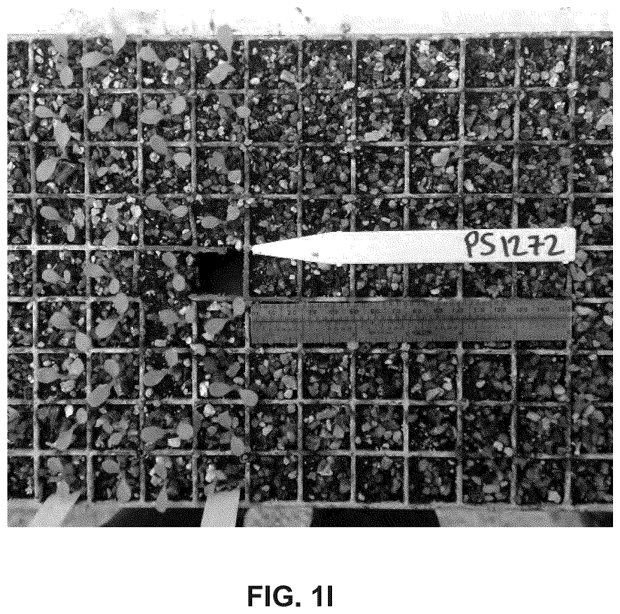

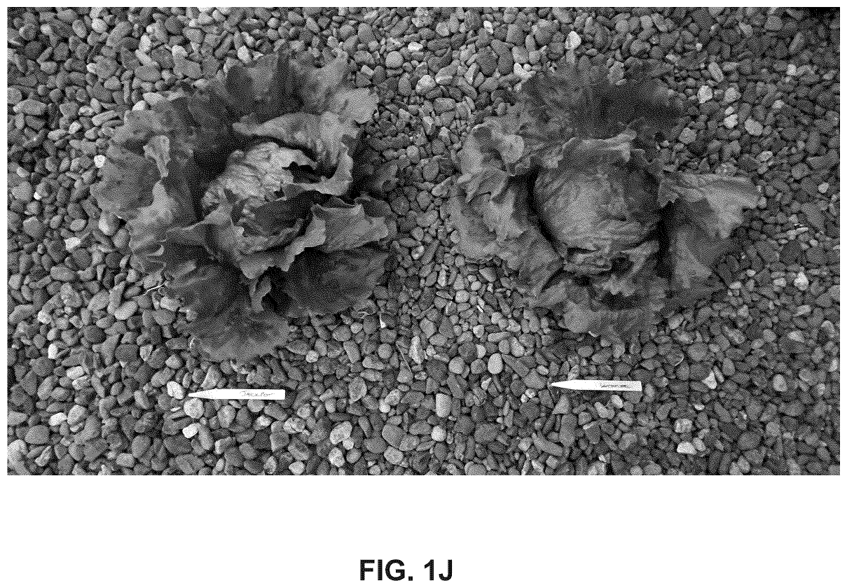

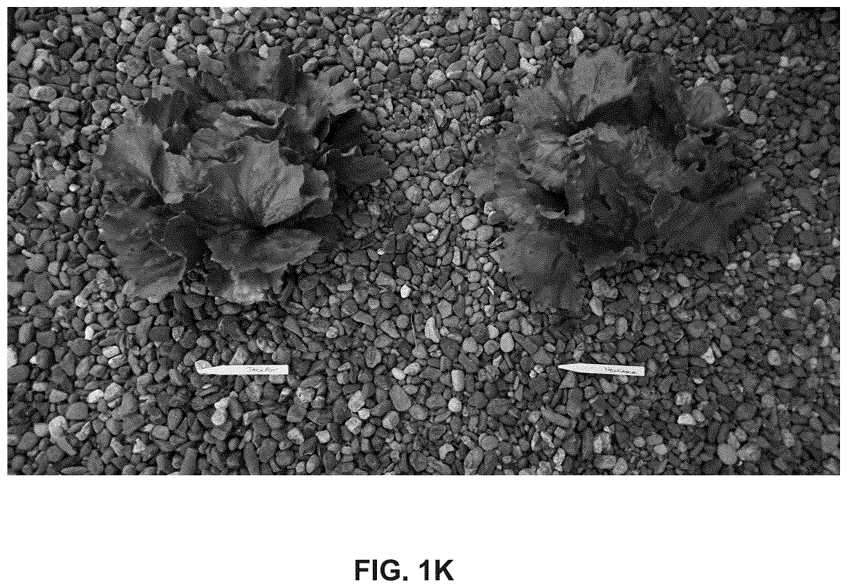

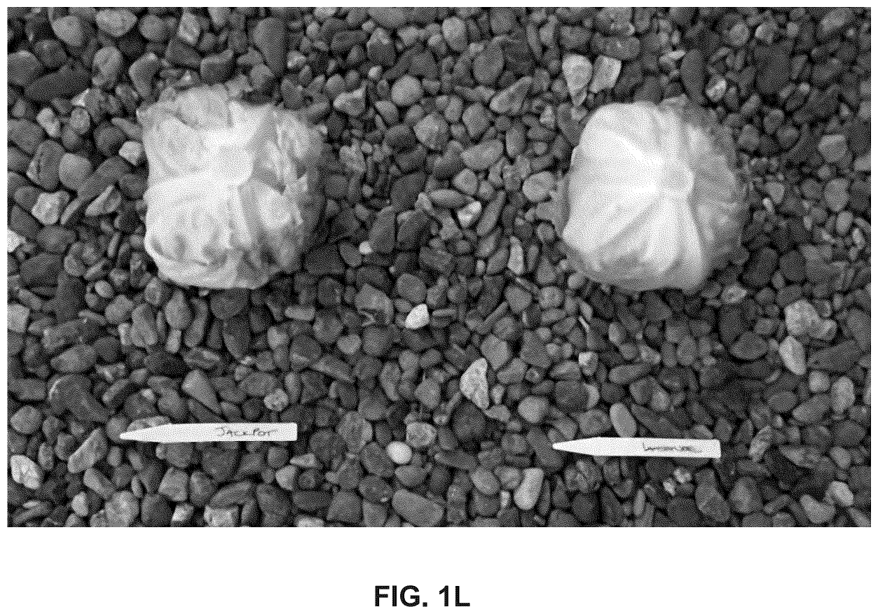

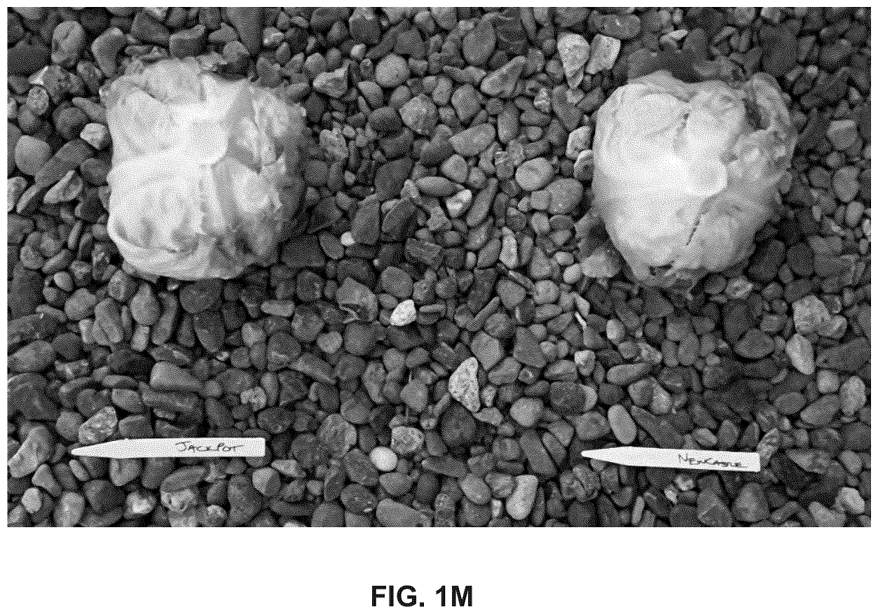

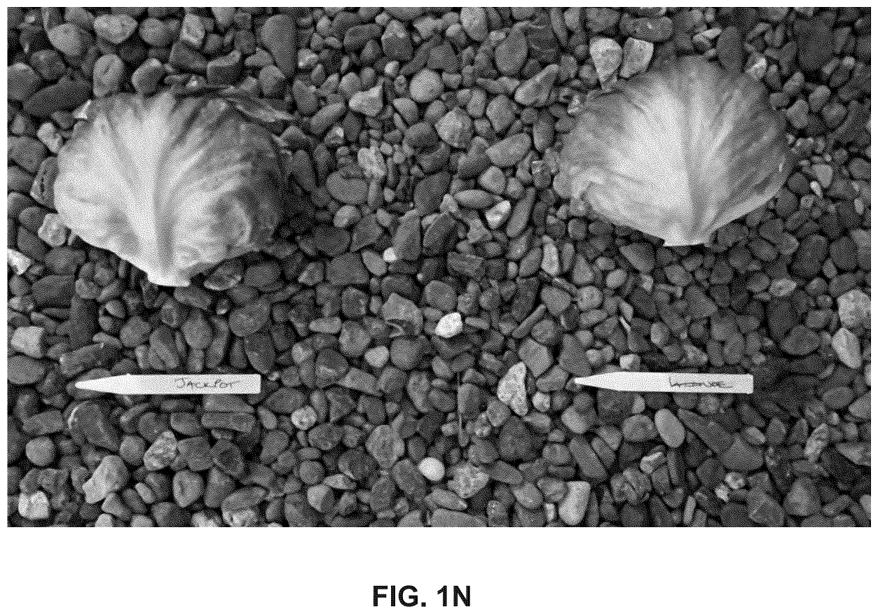

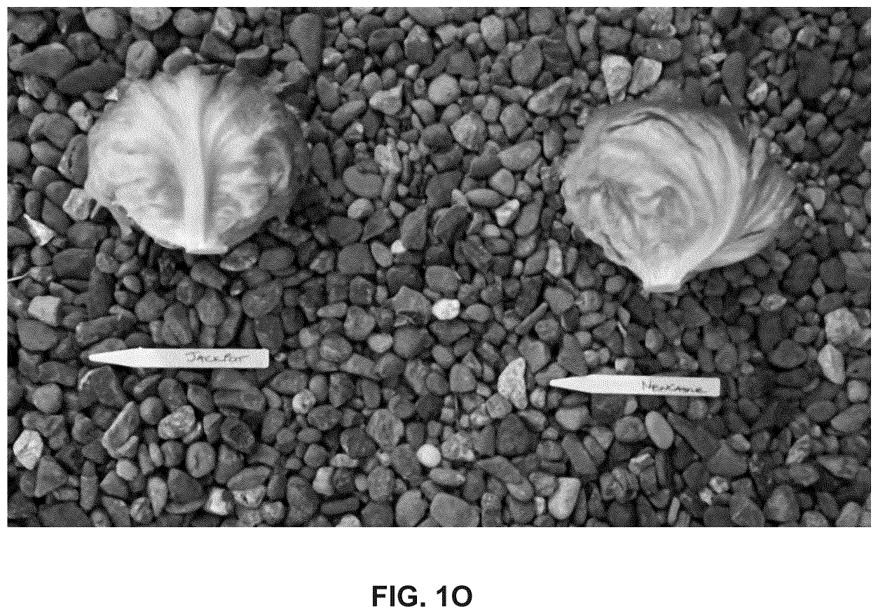

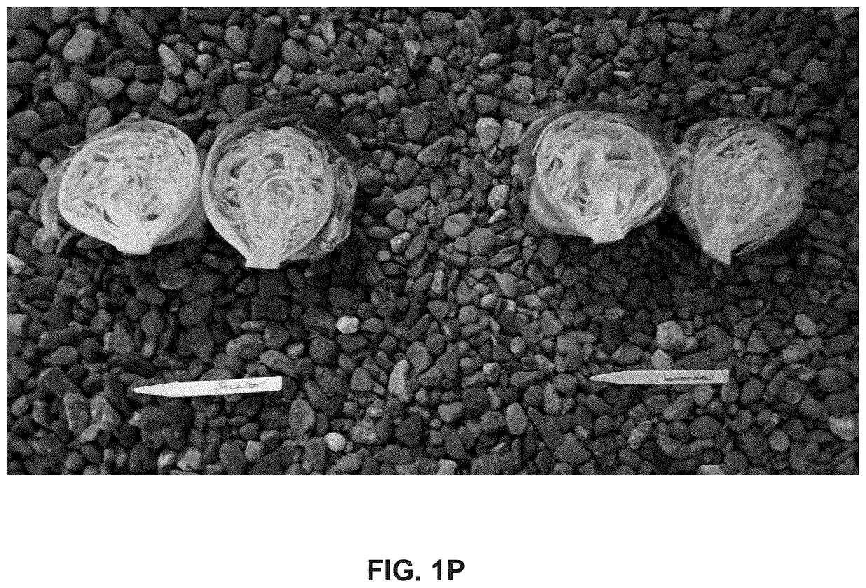

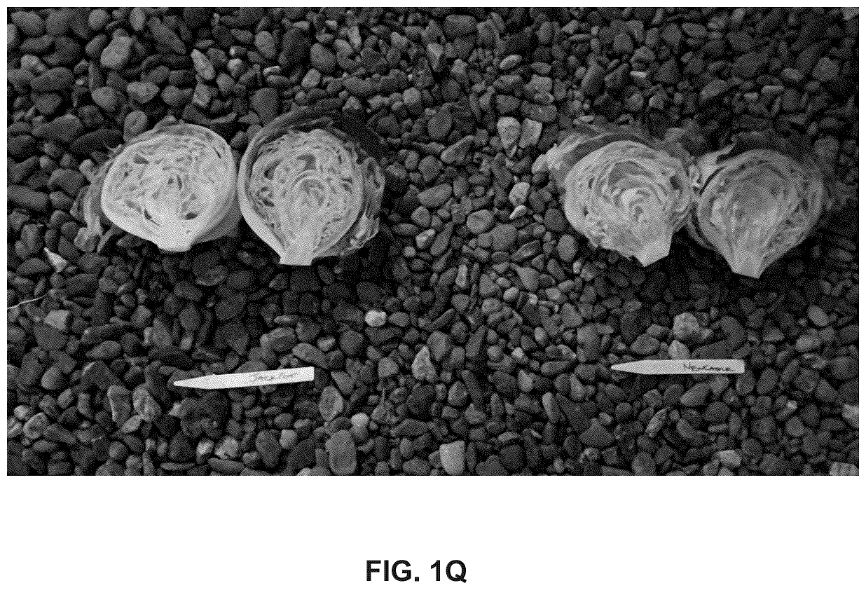

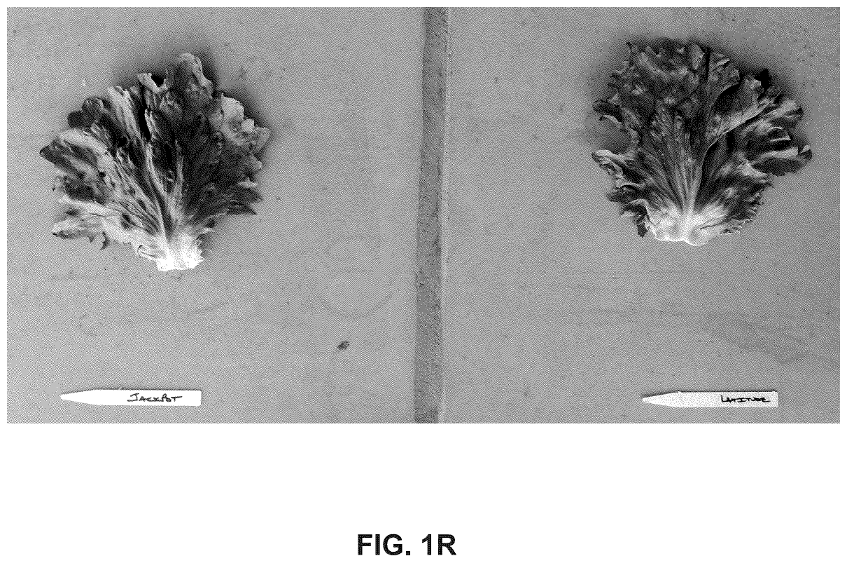

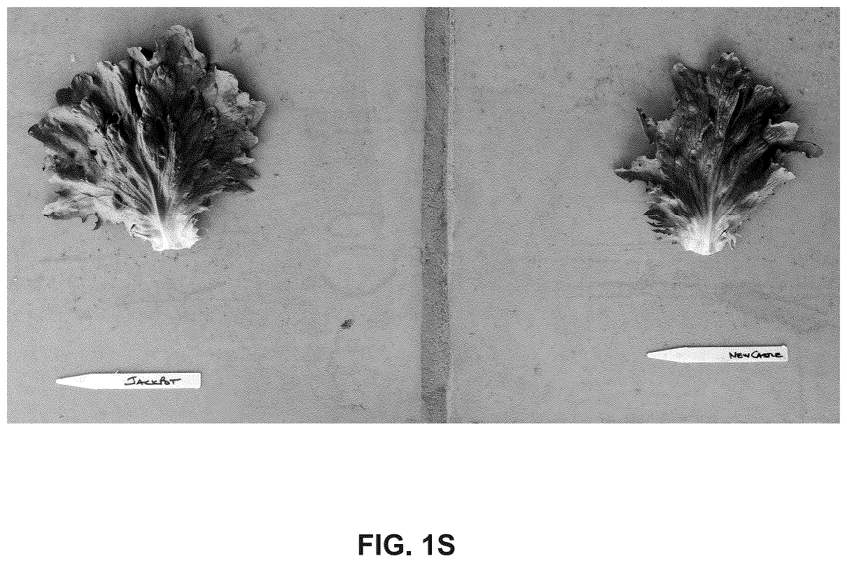

Further distinguishing features are apparent from the comparison of the variety ‘PS 1540’ with the varieties ‘Latitude’ and ‘Newcastle’ depicted in FIGS. 1A-1S.

### Objective Description of the Variety ‘PS 1541’

‘PS 1541’ is an inbred iceberg lettuce variety. This variety is distinct and unique to all other iceberg lettuce varieties due to its Fusarium resistance, Verticillium resistance, and INSV resistance. When compared to reference varieties, ‘PS 1541’ has a larger frame length, larger head diameter, and larger core diameter. ‘PS 1541’ is adapted to growing in regions such as the Southwest and West Coast regions of the United States, such as California and the Arizona desert. In particular, ‘PS 1541’ is suitable for growing in the spring for areas such as Yuma, Arizona, USA, and in the summer for areas such as Salinas, California, USA. Lettuce variety ‘PS 1541’ is the result of numerous generations of plant selections chosen for its Fusarium resistance, Verticillium resistance, and INSV resistance.

The variety has shown uniformity and stability for the traits, within the limits of environmental influence for the traits. It has been self-pollinated a sufficient number of generations with careful attention to uniformity of plant type. The line has been increased with continued observation for uniformity. No variant traits have been observed or are expected in variety ‘PS 1541’.
Lettuce variety ‘PS 1541’ has the following morphologic and other characteristics:
Plant type: Crisp (i.e., iceberg)
Seed:
Color: White (silver gray)
Light dormancy: Light not required
Heat dormancy: Susceptible
Cotyledon to Fourth Leaf Stage:
Shape of cotyledons: Spatulate
Cotyledon length: 12.2 mm
Cotyledon width: 7.1 mm
Cotyledon index (length/width×10): 17.2
Shape of fourth leaf: Elongated
Fourth leaf length: 23.8 mm
Fourth leaf width: 59.3 mm
Fourth leaf index (length/width×10): 4
Apical margin: Finely dentate
Basal margin: Finely dentate
Green color: Dark green
Anthocyanin distribution: Absent
Cupping: Uncupped
Reflexing: None
Mature Leaves:
Margin:
Incision depth (deepest penetration of the margin): Moderate
Indentation (finest divisions of the margin): Crenate
Undulation of apical margin: Moderate
Color (Munsell): 5GY 5/8 (Dark green)
Anthocyanin distribution: Absent
Glossiness: Moderate
Blistering: Absent/slight
Thickness: Intermediate
Trichomes: Absent (smooth)
Plant:
Weight: 641.4 g
Spread of frame leaves: 48.9 cm
Head diameter (market trimmed with single cap leaf): 14.3 cm
Head Shape: Spherical
Head size class: Large
Head firmness: Firm
Butt:
Shape: Rounded
Midrib: Moderately Raised
Plant Core:
Diameter at base of head: 30.8 mm
Ratio of head diameter/core diameter: 4.6
Core height from base to head to apex: 45.5 mm
Bolting:
Number of days from first water to seed stalk emergence under summer conditions: 65
Bolting class: Medium
Mature seed stalk height: 103 cm
Mature seed stalk spread: 30.2 cm
Bolter leaves: Curved
Margin: Entire
Bolter habit:
Terminal inflorescence: Present
Lateral shoots: Present
Basal side shoots: Absent
Disease/Pest Resistance:
Downy Mildew (Bremia lactucae) (Bl): Intermediate Resistant
Verticillium Race 1: Highly Resistant
Fusarium Wilt Race 1: Intermediate Resistant
INSV: Intermediate Resistant
Pythium: Intermediate Resistant
Nasonovia aphid (Nasonovia ribisnigri) biotype 0 (Nr:0): Susceptible
Stress Resistance:
Tipburn: Intermediate tip burn resistance

### Comparisons to Commercial Lettuce Variety

Table 7 below compares characteristics of lettuce variety ‘PS 1541’ with the lettuce variety ‘Latitude’. Column 1 lists the characteristics, column 2 shows the characteristics for lettuce variety ‘PS 1541’, column 3 shows the characteristics for lettuce variety ‘Latitude’, and column 4 shows characteristics for lettuce variety ‘Newcastle’.

TABLE 7

| Characteristic | ‘PS 1541’ | | ‘Latitude’ | | ‘Newcastle’ | |
|---|---|---|---|---|---|---|
| Seed Color | White | | Black | | Black | |
| | (Silver gray) | | | | | |
| Verticillium Resistance | Highly | | Resistant | | Highly | |
| | resistant | | | | resistant | |
| Fusarium Wilt Race 1 | Intermediate | | Susceptible | | — | |
| Resistance | Resistant | | | | | |
| INSV Resistance | Intermediate | | Highly | | — | |
| | Resistant | | Resistant | | | |
| Frame Length | 48.9 | cm | 44.9 | cm | 45.8 | cm |
| Weight | 641.4 | g | 650.5 | g | 582.8 | g |
| Head Diameter | 14.3 | mm | 13.3 | mm | 13.1 | mm |
| Core Diameter | 30.8 | mm | 29.3 | mm | 30.2 | mm |
| Core Length | 45.5 | mm | 38.9 | mm | 34.6 | mm |

| | | | |
|---|---|---|---|
| Color (Munsell) | 5GY 5/8 | 5GY 5/6 | 2.5 GY 5/6 |

Tables 8A-8C below show results of a first trial that compares the head weight, head diameter, core length, core diameter, and frame diameter of 20 plants of the lettuce variety ‘PS 1541’ (Table 8A) with those of 20 plants of lettuce variety ‘Latitude’ (Table 8B) and 20 plants of lettuce variety ‘Newcastle’ (Table 8C). The head weights shown are total head weights.

TABLE 8A

| | | Head | Core | Core | Frame |
|---|---|---|---|---|---|
| | Head Wt. | Diameter | Length | Diameter | Diameter |
| ‘PS 1541’ | (g) | (cm) | (mm) | (mm) | (cm) |
| Max | 821 | 16.3 | 55 | 36 | 53.2 |
| Min | 519 | 13.9 | 38 | 27 | 49.5 |
| Average | 646.3 | 14.9 | 49.1 | 32.9 | 51.1 |
| Std. Dev. | 112.9 | 0.8636 | 4.605 | 2.540 | 1.143 |

TABLE 8B

| | | Head | Core | Core | Frame |
|---|---|---|---|---|---|
| | Head Wt. | Diameter | Length | Diameter | Diameter |
| ‘Latitude’ | (g) | (cm) | (mm) | (mm) | (cm) |
| Max | 975 | 15.6 | 59 | 38 | 50.7 |
| Min | 558 | 12.1 | 34 | 30 | 44.1 |
| Average | 738.1 | 13.8 | 46 | 34.5 | 47.5 |
| Std. Dev. | 118.2 | 1.031 | 5.885 | 2.260 | 1.921 |

TABLE 8C

| | | Head | Core | Core | Frame |
|---|---|---|---|---|---|
| | Head Wt. | Diameter | Length | Diameter | Diameter |
| ‘Newcastle’ | (g) | (cm) | (mm) | (mm) | (cm) |
| Max | 952 | 16.6 | 44 | 35 | 49.8 |
| Min | 504 | 11.2 | 33 | 29 | 43.2 |
| Average | 631.4 | 14.0 | 37 | 32.3 | 46.8 |
| Std. Dev. | 102.4 | 1.297 | 3.026 | 1.832 | 1.744 |

Tables 9A-9C below show results of a second trial that compares the head weight, head diameter, core length, core diameter, and frame diameter of 20 plants of the lettuce variety ‘PS 1541’ (Table 9A) with those of 20 plants of lettuce variety ‘Latitude’ (Table 9B) and 20 plants of lettuce variety ‘Newcastle’ (Table 9C). The head weights shown are total head weights.

TABLE 9A

| | | Head | Core | Core | Frame |
|---|---|---|---|---|---|
| | Head Wt. | Diameter | Length | Diameter | Diameter |
| ‘PS 1541’ | (g) | (cm) | (mm) | (mm) | (cm) |
| Max | 1065 | 16.4 | 56 | 33 | 48.6 |
| Min | 571 | 11.7 | 23 | 22 | 41.6 |
| Average | 776.6 | 14.2 | 38.4 | 26.8 | 45.9 |
| Std. Dev. | 155.8 | 1.004 | 7.768 | 2.397 | 2.017 |

TABLE 9B

| | | Head | Core | Core | Frame |
|---|---|---|---|---|---|
| | Head Wt. | Diameter | Length | Diameter | Diameter |
| ‘Latitude’ | (g) | (cm) | (mm) | (mm) | (cm) |
| Max | 1060 | 15.5 | 47 | 35 | 45.6 |
| Min | 554 | 11.6 | 23 | 20 | 38.4 |
| Average | 837.4 | 13.8 | 32.3 | 26 | 42.1 |
| Std. Dev. | 150.5 | 0.9550 | 5.555 | 3.934 | 1.963 |

TABLE 9C

| | | Head | Core | Core | Frame |
|---|---|---|---|---|---|
| | Head Wt. | Diameter | Length | Diameter | Diameter |
| ‘Newcastle’ | (g) | (cm) | (mm) | (mm) | (cm) |
| Max | 954 | 14.3 | 34 | 38 | 48.4 |
| Min | 511 | 8.6 | 18 | 22 | 39.4 |
| Average | 682.9 | 12.5 | 24.8 | 27.8 | 42.8 |
| Std. Dev. | 111.8 | 1.384 | 3.492 | 3.365 | 2.146 |

Tables 10A-10C below show results of a third trial that compares the head weight, head diameter, core length, core diameter, and frame diameter of 20 plants of the lettuce variety ‘PS 1541’ (Table 10A) with those of 20 plants of lettuce variety ‘Latitude’ (Table 10B) and 20 plants of lettuce variety ‘Newcastle’ (Table 10C). The head weights shown are total head weights.

TABLE 10A

| | | Head | Core | Core | Frame |
|---|---|---|---|---|---|
| | Head Wt. | Diameter | Length | Diameter | Diameter |
| ‘PS 1541’ | (g) | (cm) | (mm) | (mm) | (cm) |
| Max | 735 | 15.3 | 54 | 37 | 55.3 |
| Min | 434 | 11.8 | 35 | 24 | 44.2 |
| Average | 568 | 13.7 | 44.1 | 32 | 49.1 |
| Std. Dev. | 98.26 | 1.072 | 5.671 | 2.956 | 2.893 |

TABLE 10B

| | | Head | Core | Core | Frame |
|---|---|---|---|---|---|
| | Head Wt. | Diameter | Length | Diameter | Diameter |
| ‘Latitude’ | (g) | (cm) | (mm) | (mm) | (cm) |
| Max | 759 | 14.7 | 39 | 34 | 50.3 |
| Min | 433 | 10.2 | 27 | 26 | 44.3 |
| Average | 598.8 | 12.6 | 33.9 | 30.6 | 46.9 |
| Std. Dev. | 101.2 | 1.327 | 3.249 | 2.139 | 1.583 |

TABLE 10C

| | Head | Head | Core | Core | Frame |
|---|---|---|---|---|---|
| | Wt. | Diameter | Length | Diameter | Diameter |
| ‘Newcastle’ | (g) | (cm) | (mm) | (mm) | (cm) |
| Max | 739 | 14.9 | 36 | 36 | 52.1 |
| Min | 352 | 10.2 | 19 | 25 | 44.3 |
| Average | 535.4 | 12.4 | 27.3 | 30.8 | 48.4 |
| Std. Dev. | 89.41 | 1.077 | 4.354 | 2.845 | 2.375 |

Tables 11A-11C below show results of a fourth trial that compares the head weight, head diameter, core length, core diameter, and frame diameter of 20 plants of the lettuce variety ‘PS 1541’ (Table 11A) with those of 20 plants of lettuce variety ‘Latitude’ (Table 11B) and 20 plants of lettuce variety ‘Newcastle’ (Table 11C). The head weights shown are total head weights.

TABLE 11A

| | | Head | Core | Core | |
|---|---|---|---|---|---|
| | Head Wt. | Diameter | Length | Diameter | Frame |
| ‘PS 1541’ | (g) | (cm) | (mm) | (mm) | Diameter (cm) |
| Max | 694.5 | 15.9 | 61 | 34 | 54.8 |
| Min | 445.2 | 12.3 | 42 | 27 | 45.7 |
| Average | 574.8 | 14.2 | 50.4 | 31.6 | 49.7 |
| Std. Dev. | 76.97 | 1.076 | 5.343 | 1.701 | 2.187 |

TABLE 11B

| | | Head | Core | Core | |
|---|---|---|---|---|---|
| | Head Wt. | Diameter | Length | Diameter | Frame |
| ‘Latitude’ | (g) | (cm) | (mm) | (mm) | Diameter (cm) |
| Max | 623.4 | 14.5 | 57 | 29 | 49.7 |
| Min | 261.5 | 11.1 | 31 | 22 | 37.6 |
| Average | 427.7 | 13.0 | 43.6 | 26.1 | 43.0 |
| Std. Dev. | 86.91 | 1.071 | 5.790 | 1.791 | 3.766 |

TABLE 11C

| | Head | Head | Core | Core | Frame |
|---|---|---|---|---|---|
| | Wt. | Diameter | Length | Diameter | Diameter |
| ‘Newcastle’ | (g) | (cm) | (mm) | (mm) | (cm) |
| Max | 692.6 | 15.8 | 61 | 33 | 53.4 |
| Min | 315.7 | 11.8 | 35 | 26 | 41.2 |
| Average | 481.5 | 13.6 | 49.3 | 29.9 | 45.4 |
| Std. Dev. | 109.9 | 0.9544 | 8.117 | 2.100 | 3.013 |

Tables 12A-12C below compares the fourth leaf width, fourth leaf length, cotyledon leaf width, cotyledon leaf length, stalk length, and frame of 20 plants of the lettuce variety ‘PS 1541’ (Table 12A) with those of 20 plants of lettuce variety ‘Latitude’ (Table 12B) and 20 plants of lettuce variety ‘Newcastle’ (Table 12C). The head weights shown are total head weights.

TABLE 12A

| | Fourth | Fourth | Cot. | | | |
|---|---|---|---|---|---|---|
| | leaf | leaf | leaf | Cot. leaf | | |
| | width | length | width | length | Stalk | Frame |
| ‘PS 1541’ | (mm) | (mm) | (mm) | (mm) | (cm) | (cm) |
| Max | 71 | 27 | 8 | 15 | 114.3 | 35.6 |
| Min | 50 | 20 | 6 | 10 | 86.5 | 25.7 |
| Average | 59.3 | 23.8 | 7.1 | 12.2 | 103 | 30.2 |
| Std. Dev. | 6.632 | 1.673 | 0.7881 | 1.309 | 6.812 | 3.040 |

TABLE 12B

| | Fourth | Fourth | Cot. | | | |
|---|---|---|---|---|---|---|
| | leaf | leaf | leaf | Cot. leaf | | |
| | width | length | width | length | Stalk | Frame |
| ‘Latitude’ | (mm) | (mm) | (mm) | (mm) | (cm) | (cm) |
| Max | 71 | 28 | 9 | 14 | 108.4 | 33.2 |
| Min | 50 | 22 | 6 | 9 | 86.5 | 23.2 |
| Average | 60.6 | 24.2 | 7.5 | 11.7 | 98.4 | 28.7 |
| Std. Dev. | 5.256 | 1.631 | 0.7592 | 1.565247584 | 6.134 | 2.098 |

TABLE 12C

| | Fourth | Fourth | Cot. | | | |
|---|---|---|---|---|---|---|
| | leaf | leaf | leaf | Cot. leaf | | |
| | width | length | width | length | Stalk | Frame |
| ‘Newcastle’ | (mm) | (mm) | (mm) | (mm) | (cm) | (cm) |
| Max | 57 | 23 | 8 | 15 | 104.2 | 29.4 |
| Min | 43 | 20 | 5 | 9 | 85.7 | 22.8 |
| Average | 51.4 | 21.2 | 7.2 | 12 | 93.5 | 25.9 |
| Std. Dev. | 3.858 | 1.056 | 0.8127 | 1.525 | 5.235 | 1.781 |

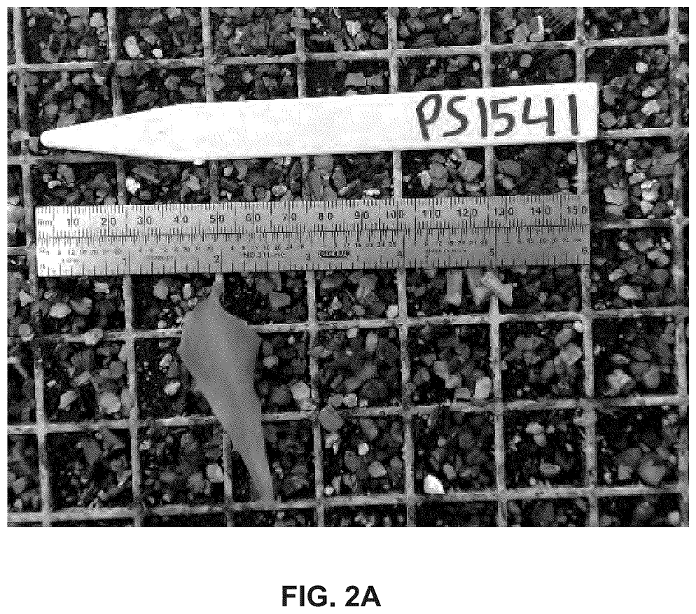

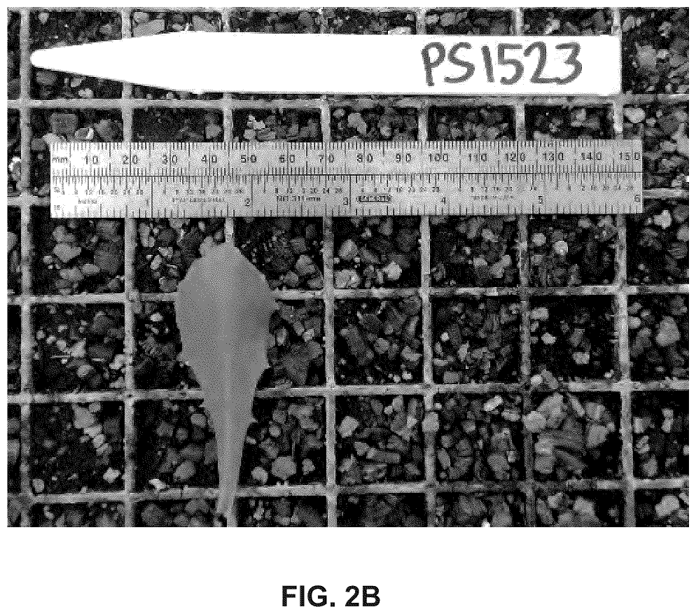

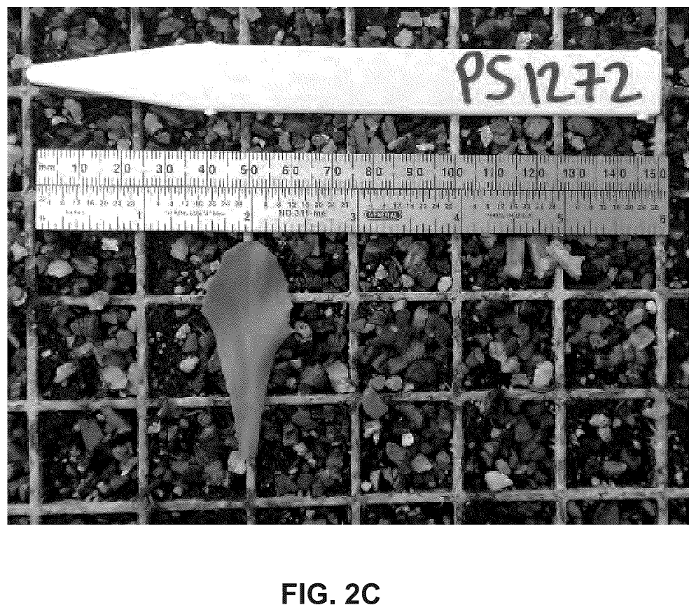

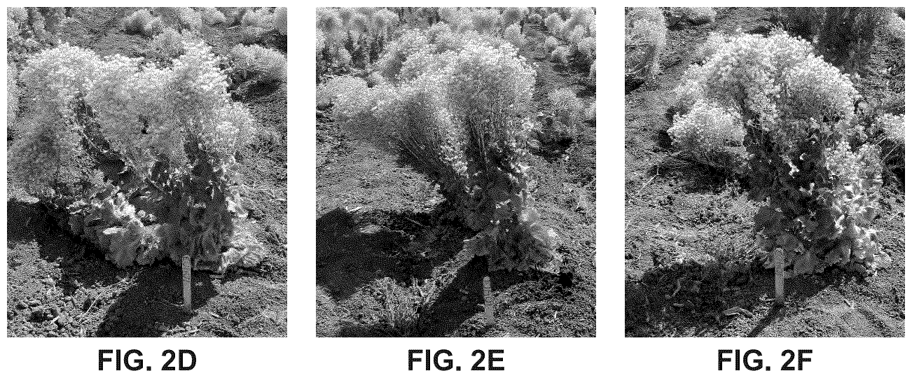

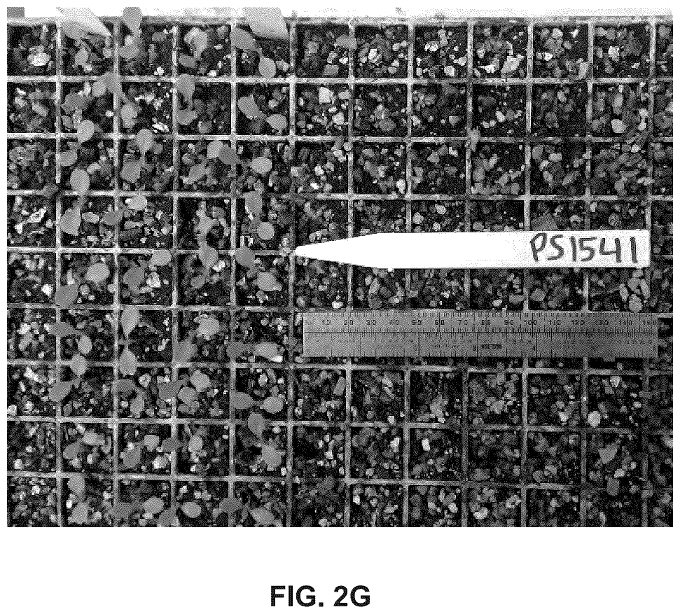

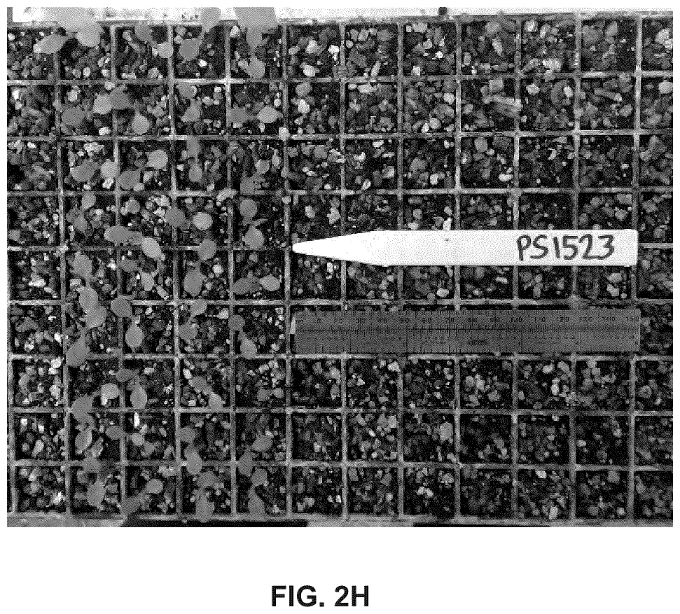

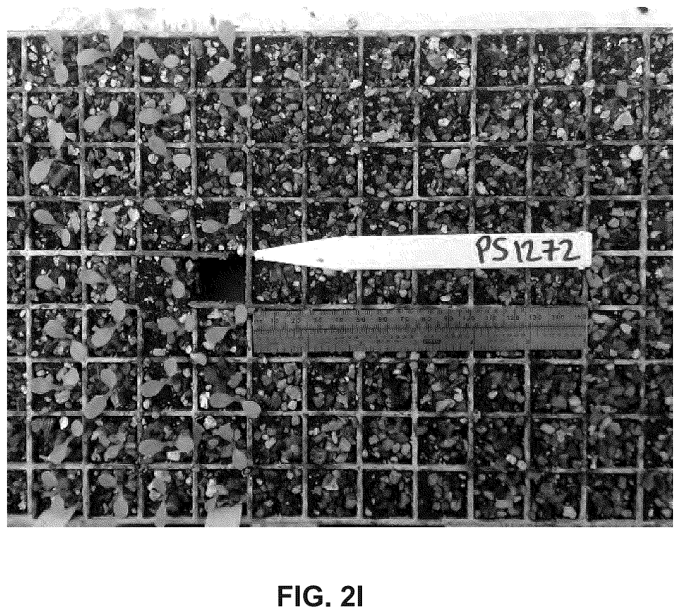

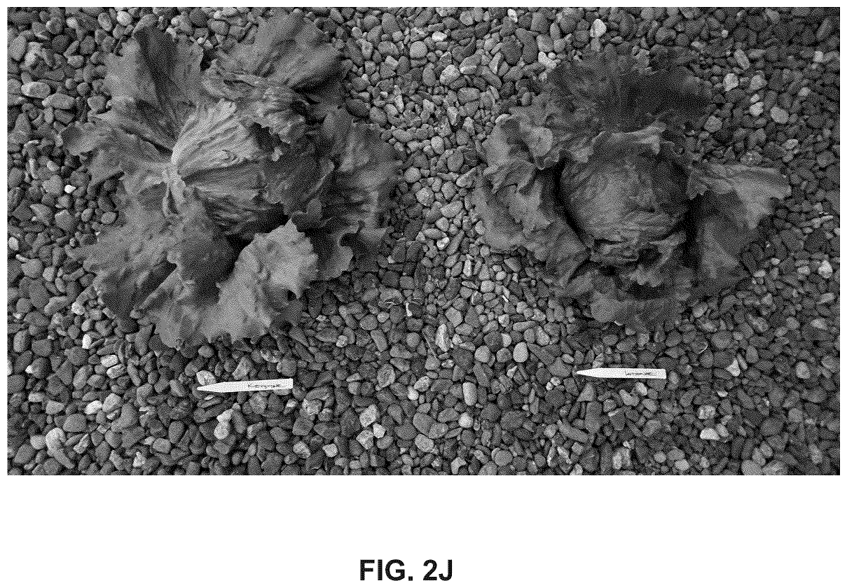

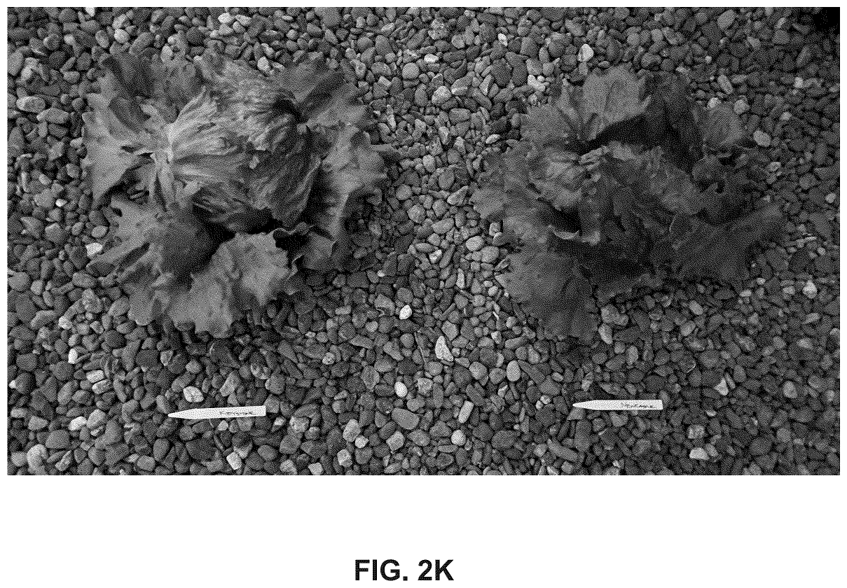

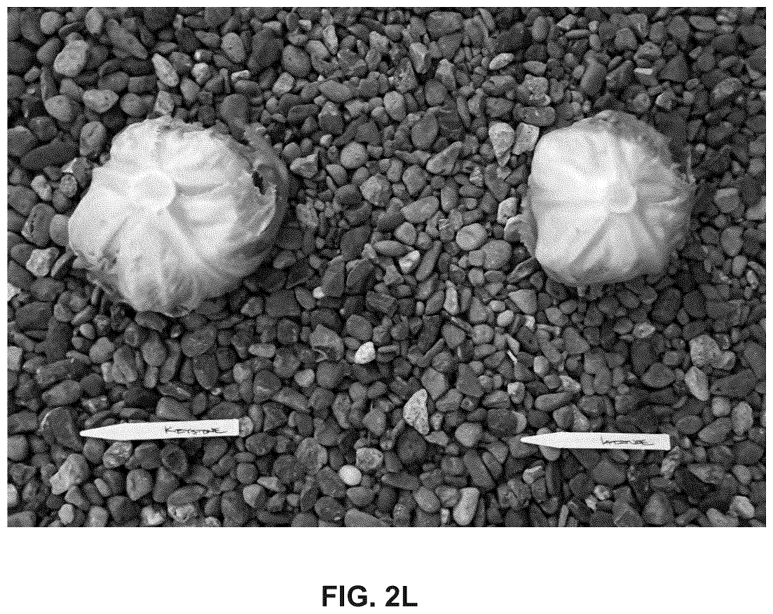

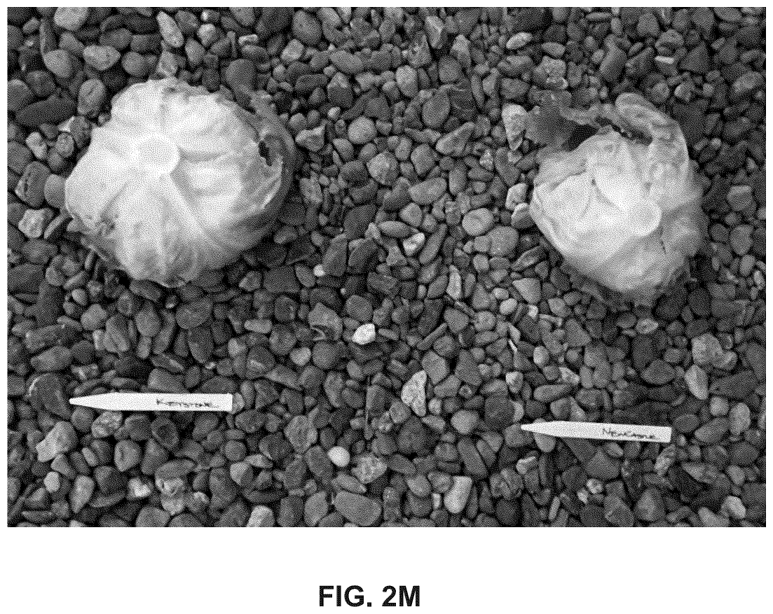

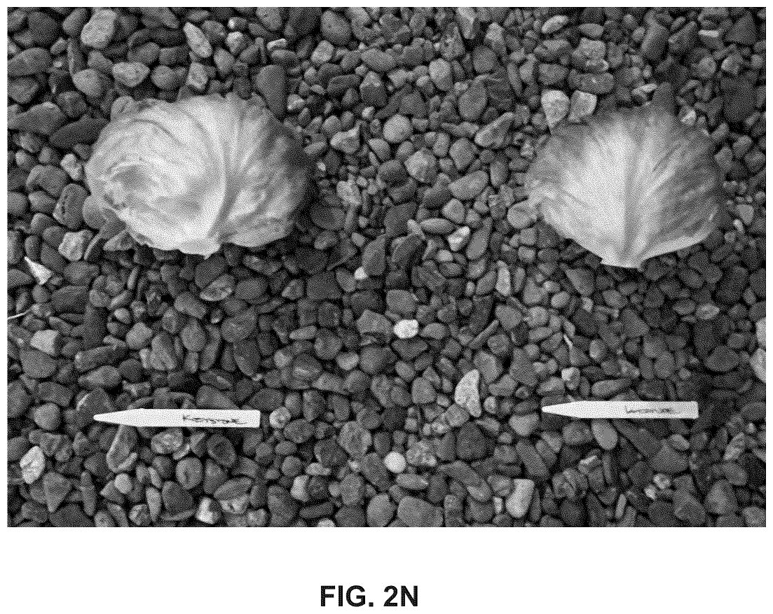

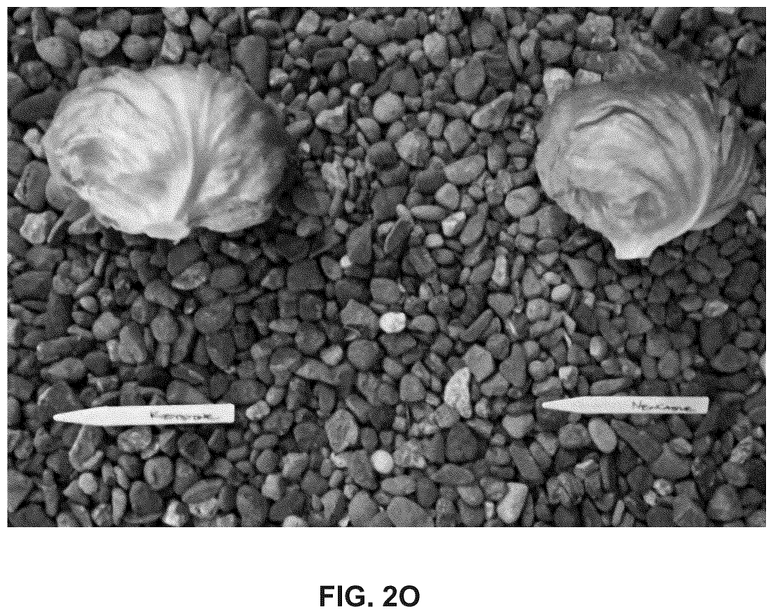

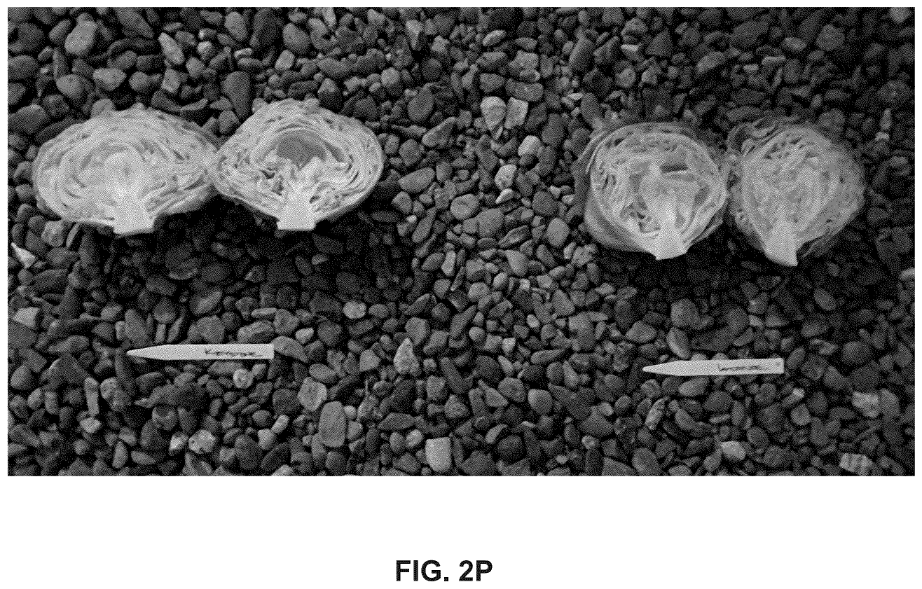

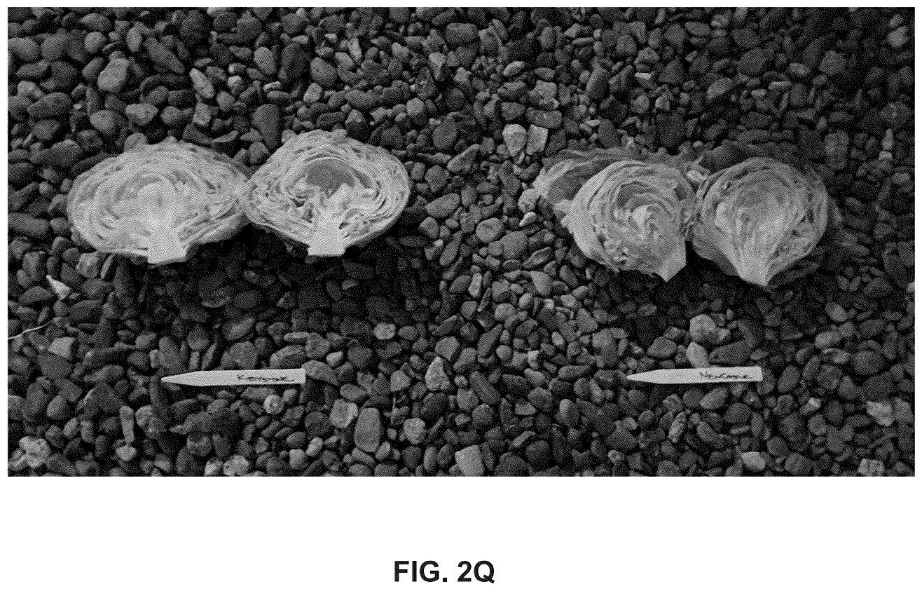

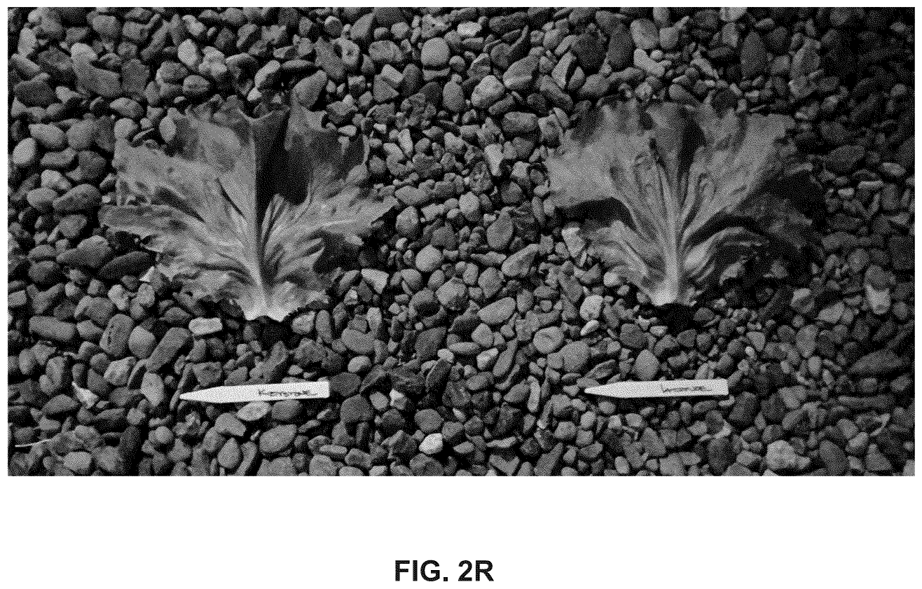

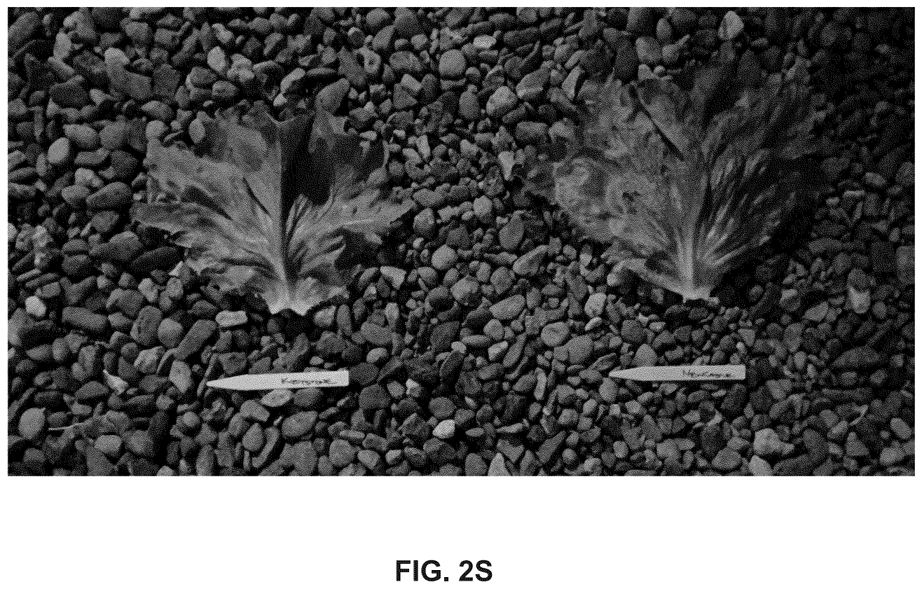

Further distinguishing features are apparent from the comparison of the variety ‘PS 1541’ with the varieties ‘Latitude’ and ‘Newcastle’ depicted in FIGS. 2A-2S.

### Objective Description of the Variety ‘PS 1546’

‘PS 1546’ is an inbred iceberg lettuce variety. This variety is distinct and unique to all other iceberg lettuce varieties due to its Fusarium resistance and INSV resistance. When compared to reference varieties, ‘PS 1546’ has a larger frame length, increased weight, larger head diameter, and larger core diameter. ‘PS 1546’ is adapted to growing in regions such as the Southwest and West Coast regions of the United States, such as California and the Arizona desert. In particular, ‘PS 1546’ is suitable for growing in the spring for areas such as Yuma, Arizona, USA, and in the summer for areas such as Salinas, California, USA. Lettuce variety ‘PS 1546’ is the result of numerous generations of plant selections chosen for its Fusarium resistance and INSV resistance.

The variety has shown uniformity and stability for the traits, within the limits of environmental influence for the traits. It has been self-pollinated a sufficient number of generations with careful attention to uniformity of plant type. The line has been increased with continued observation for uniformity. No variant traits have been observed or are expected in variety ‘PS 1546’.
Lettuce variety ‘PS 1546’ has the following morphologic and other characteristics:
Plant type: Crisp (i.e., iceberg)
Seed:
Color: White (silver gray)
Light dormancy: Light not required
Heat dormancy: Susceptible
Cotyledon to Fourth Leaf Stage:
Shape of cotyledons: Spatulate
Cotyledon length: 12.9 mm
Cotyledon width: 7.2 mm
Cotyledon index (length/width×10): 17.9
Shape of fourth leaf: Elongated
Fourth leaf length: 24.7 mm
Fourth leaf width: 62.8 mm
Fourth leaf index (length/width×10): 3.9
Apical margin: Finely dentate
Basal margin: Finely dentate
Green color: Dark green
Anthocyanin distribution: Absent
Cupping: Slight
Reflexing: None
Mature Leaves:
Margin:
Incision depth (deepest penetration of the margin): Moderate
Indentation (finest divisions of the margin): Crenate
Undulation of apical margin: Moderate
Color (Munsell): 5GY 5/6 (Dark green)
Anthocyanin distribution: Absent
Glossiness: Dull
Blistering: Moderate
Thickness: Intermediate
Trichomes: Absent (smooth)
Plant:
Weight: 682.3 g
Spread of frame leaves: 44.4 cm
Head diameter (market trimmed with single cap leaf): 14.1 cm
Head Shape: Spherical
Head size class: Large
Head firmness: Firm
Butt:
Shape: Rounded
Midrib: Moderately Raised
Plant Core:
Diameter at base of head: 32.4 mm
Ratio of head diameter/core diameter: 4.4
Core height from base to head to apex: 48.9 mm
Bolting:
Number of days from first water to seed stalk emergence under summer conditions: 63
Bolting class: Medium
Mature seed stalk height: 84.5 cm
Mature seed stalk spread: 25.4 cm
Bolter leaves: Curved
Margin: Entire
Bolter habit:
Terminal inflorescence: Present
Lateral shoots: Present
Basal side shoots: Absent
Disease Resistance:
Downy Mildew: Intermediate Resistance
Fusarium Wilt Race 1: Resistant
INSV: Resistant
Stress Resistance:
Tipburn: Intermediate tip burn resistance

### Comparisons to Commercial Lettuce Variety

Table 13 below compares characteristics of lettuce variety ‘PS 1546’ with the lettuce varieties ‘Meridian’ (U.S. Pat. No. 9,706,743) and ‘Salute’ (U.S. Pat. No. 8,835,719). Column 1 lists the characteristics, column 2 shows the characteristics for lettuce variety ‘PS 1546’, column 3 shows the characteristics for lettuce variety ‘Meridian’, and column 4 shows the characteristics for lettuce variety ‘Salute’.

TABLE 13

| | | | |
|---|---|---|---|
| Characteristic | ‘PS 1546’ | ‘Meridian’ | ‘Salute’ |

| | | | |
|---|---|---|---|
| Seed | Susceptible | Absent | Yes |

| | | | | | | |
|---|---|---|---|---|---|---|
| Heat dormancy | | | | | | |
| Frame Length | 44.4 | cm | 41.9 | cm | 43.7 | cm |
| Weight | 682.3 | g | 654 | g | 509 | g |
| Head Diameter | 14.1 | mm | 12.6 | mm | 11.6 | mm |
| Core Diameter | 32.4 | mm | 30 | mm | 29.1 | mm |
| Core Length | 48.9 | mm | 36.5 | mm | 23.5 | mm |

| | | | |
|---|---|---|---|
| Color (Munsell) | 5GY 5/6 | 5GY 5/6 | 5GY 4/8 |

Tables 14A-14C below show results of a first trial that compares the head weight, head diameter, core length, core diameter, and frame diameter of 20 plants of the lettuce variety ‘PS 1546’ (Table 14A) with those of 20 plants of lettuce variety ‘Meridian’ (Table 14B) and 20 plants of lettuce variety ‘Salute’ (Table 14C). The head weights shown are total head weights.

TABLE 14A

| | | Head | Core | Core | |
|---|---|---|---|---|---|
| | Head Wt. | Diameter | Length | Diameter | Frame |
| ‘PS 1546’ | (g) | (cm) | (mm) | (mm) | Diameter (cm) |
| Max | 968 | 16.7 | 49 | 37 | 48.6 |
| Min | 518 | 12.7 | 19 | 21 | 36.8 |
| Average | 794.3 | 14.9 | 38.3 | 29.2 | 43.4 |
| Std. Dev. | 122.5 | 1.054 | 7.240 | 4.511 | 2.668 |

TABLE 14B

| | | Head | Core | Core | |
|---|---|---|---|---|---|
| | Head Wt. | Diameter | Length | Diameter | Frame |
| ‘Meridian’ | (g) | (cm) | (mm) | (mm) | Diameter (cm) |
| Max | 933 | 14.3 | 35 | 32 | 44.6 |
| Min | 543 | 10.2 | 24 | 21 | 38.7 |
| Average | 748.7 | 12.0 | 29.5 | 26.7 | 42.2 |
| Std. Dev. | 114.1 | 0.9506 | 3.364 | 3.117 | 1.715 |

TABLE 14C

| | | Head | Core | Core | |
|---|---|---|---|---|---|
| | Head Wt. | Diameter | Length | Diameter | Frame |
| ‘Salute’ | (g) | (cm) | (mm) | (mm) | Diameter (cm) |
| Max | 921 | 16 | 32 | 38 | 49.4 |
| Min | 441 | 11.5 | 18 | 22 | 41.6 |
| Average | 684 | 13.5 | 23.8 | 31.5 | 46.2 |
| Std. Dev. | 130.2 | 0.9981 | 4.552 | 3.720 | 2.442 |

Tables 15A-15C below show results of a second trial that compares the head weight, head diameter, core length, core diameter, and frame diameter of 20 plants of the lettuce variety ‘PS 1546’ (Table 15A) with those of 20 plants of lettuce variety ‘Meridian’ (Table 15B) and 20 plants of lettuce variety ‘Salute’ (Table 15C). The head weights shown are total head weights.

TABLE 15A

| | | Head | Core | Core | |
|---|---|---|---|---|---|
| | Head Wt. | Diameter | Length | Diameter | Frame |
| ‘PS 1546’ | (g) | (cm) | (mm) | (mm) | Diameter (cm) |
| Max | 629 | 14.5 | 54 | 36 | 48.2 |
| Min | 369 | 8.6 | 29 | 26 | 43.2 |
| Average | 493.1 | 12.9 | 36.9 | 32.6 | 45.7 |
| Std. Dev. | 82.82 | 1.426 | 5.412 | 2.645 | 1.441 |

TABLE 15B

| | | Head | Core | Core | |
|---|---|---|---|---|---|
| | Head Wt. | Diameter | Length | Diameter | Frame |
| ‘Meridian’ | (g) | (cm) | (mm) | (mm) | Diameter (cm) |
| Max | 670 | 13.6 | 44 | 33 | 49.2 |
| Min | 385 | 10.2 | 24 | 27 | 36.1 |
| Average | 516.2 | 11.3 | 32.1 | 30.3 | 40.9 |
| Std. Dev. | 78.70 | 0.9602 | 4.957 | 1.838 | 3.306 |

TABLE 15C

| | | Head | Core | Core | |
|---|---|---|---|---|---|
| | Head Wt. | Diameter | Length | Diameter | Frame |
| ‘Salute’ | (g) | (cm) | (mm) | (mm) | Diameter (cm) |
| Max | 500 | 11.6 | 24 | 32 | 48.4 |
| Min | 110 | 5.6 | 11 | 17 | 38.1 |
| Average | 269.4 | 8.8 | 16.5 | 24.5 | 43.9 |
| Std. Dev. | 127.3 | 1.685 | 3.035 | 4.174 | 3.017 |

Tables 16A-16C below show results of a third trial that compares the head weight, head diameter, core length, core diameter, and frame diameter of 20 plants of the lettuce variety ‘PS 1546’ (Table 16A) with those of 20 plants of lettuce variety ‘Meridian’ (Table 16B) and 20 plants of lettuce variety ‘Salute’ (Table 16C). The head weights shown are total head weights.

TABLE 16A

| | | Head | Core | Core | |
|---|---|---|---|---|---|
| | Head Wt. | Diameter | Length | Diameter | Frame |
| ‘PS 1546’ | (g) | (cm) | (mm) | (mm) | Diameter (cm) |
| Max | 1050 | 16.2 | 84.5 | 42.2 | 47 |
| Min | 479 | 12.4 | 37.7 | 28.2 | 37 |
| Average | 816 | 14.0 | 62.3 | 36.2 | 40.9 |
| Std. Dev. | 135.1 | 1.041 | 15.40 | 3.082 | 2.732 |

TABLE 16B

| | | Head | Core | Core | |
|---|---|---|---|---|---|
| | Head Wt. | Diameter | Length | Diameter | Frame |
| ‘Meridian’ | (g) | (cm) | (mm) | (mm) | Diameter (cm) |
| Max | 1007 | 17.1 | 86.8 | 81.2 | 46 |
| Min | 630 | 10.8 | 30.3 | 28.8 | 35 |
| Average | 825 | 13.2 | 47.1 | 35.7 | 39.9 |
| Std. Dev. | 93.77 | 1.575 | 15.07 | 10.93 | 2.845 |

TABLE 16C

| | | Head | Core | Core | |
|---|---|---|---|---|---|
| | Head Wt. | Diameter | Length | Diameter | Frame |
| ‘Salute’ | (g) | (cm) | (mm) | (mm) | Diameter (cm) |
| Max | 926 | 14.5 | 41 | 42.1 | 49 |
| Min | 592 | 11.2 | 17.8 | 28.1 | 35 |
| Average | 750.4 | 12.9 | 30.6 | 34.7 | 42 |
| Std. Dev. | 86.72 | 0.8328 | 6.254 | 3.957 | 4.146 |

Tables 17A-17C below show results of a fourth trial that compares the head weight, head diameter, core length, core diameter, and frame diameter of 20 plants of the lettuce variety

‘PS 1546’ (Table 17A) with those of 20 plants of lettuce variety ‘Meridian’ (Table 17B) and 20 plants of lettuce variety ‘Salute’ (Table 17C). The head weights shown are total head weights.

TABLE 17A

| | | Head | Core | Core | |
|---|---|---|---|---|---|
| | Head Wt. | Diameter | Length | Diameter | Frame |
| ‘PS 1546’ | (g) | (cm) | (mm) | (mm) | Diameter (cm) |
| Max | 886 | 16.8 | 79 | 35 | 51.3 |
| Min | 465 | 12.7 | 43 | 28 | 44.3 |
| Average | 626 | 14.7 | 58.3 | 31.8 | 47.6 |
| Std. Dev. | 94.69 | 1.112 | 10.78 | 2.245 | 2.154 |

TABLE 17B

| | | Head | Core | Core | |
|---|---|---|---|---|---|
| | Head Wt. | Diameter | Length | Diameter | Frame |
| ‘Meridian’ | (g) | (cm) | (mm) | (mm) | Diameter (cm) |
| Max | 658 | 16.3 | 51 | 31 | 49.4 |
| Min | 399 | 11.6 | 30 | 25 | 42.1 |
| Average | 526.3 | 14.1 | 37.5 | 27.5 | 45.0 |
| Std. Dev. | 71.94 | 1.077 | 5.236 | 1.701 | 2.215 |

TABLE 17C

| | | Head | Core | Core | |
|---|---|---|---|---|---|
| | Head Wt. | Diameter | Length | Diameter | Frame |
| ‘Salute’ | (g) | (cm) | (mm) | (mm) | Diameter (cm) |
| Max | 592 | 14.3 | 37 | 39 | 48.3 |
| Min | 192 | 7.5 | 15 | 18 | 37.4 |
| Average | 332.4 | 11.0 | 23.3 | 26.0 | 42.7 |
| Std. Dev. | 131.7 | 1.790 | 4.813 | 5.266 | 2.858 |

Tables 18A-18C below compares the fourth leaf width, fourth leaf length, cotyledon leaf width, cotyledon leaf length, stalk length, and frame of 20 plants of the lettuce variety ‘PS 1546’ (Table 18A) with those of 20 plants of lettuce variety ‘Meridian’ (Table 18B) and 20 plants of lettuce variety ‘Salute’ (Table 18C). The head weights shown are total head weights.

TABLE 18A

| | Fourth | Fourth | Cot. | | | |
|---|---|---|---|---|---|---|
| | leaf | leaf | leaf | Cot. leaf | | |
| | width | length | width | length | Stalk | Frame |
| ‘PS 1546’ | (mm) | (mm) | (mm) | (mm) | (cm) | (cm) |
| Max | 73 | 27 | 8 | 16 | 94.6 | 29.8 |
| Min | 51 | 22 | 5 | 10 | 75.4 | 22.7 |
| Average | 62.8 | 24.7 | 7.2 | 12.9 | 84.5 | 25.4 |
| Std. Dev. | 5.766 | 1.490 | 0.8127 | 1.899 | 4.830 | 1.925 |

TABLE 18B

| | Fourth | Fourth | Cot. | | | |
|---|---|---|---|---|---|---|
| | leaf | leaf | leaf | Cot. leaf | | |
| | width | length | width | length | Stalk | Frame |
| ‘Meridian’ | (mm) | (mm) | (mm) | (mm) | (cm) | (cm) |
| Max | 76 | 30 | 8 | 17 | 108.8 | 29.4 |
| Min | 46 | 21 | 6 | 12 | 88.6 | 23.6 |
| Average | 62.3 | 25.9 | 7.3 | 13.3 | 98.7 | 26.3 |
| Std. Dev. | 7.806 | 2.434 | 0.5712 | 1.410 | 5.525 | 1.786 |

TABLE 18C

| | Fourth | Fourth | Cot. | | | |
|---|---|---|---|---|---|---|
| | leaf | leaf | leaf | Cot. leaf | | |
| | width | length | width | length | Stalk | Frame |
| ‘Salute’ | (mm) | (mm) | (mm) | (mm) | (cm) | (cm) |
| Max | 70 | 27 | 8 | 19 | 121.2 | 31.6 |
| Min | 51 | 21 | 6 | 11 | 96.2 | 22.1 |
| Average | 62.5 | 24.7 | 7.6 | 14.3 | 110.9 | 27.5 |
| Std. Dev. | 5.424 | 1.922 | 0.5982 | 2.049 | 6.520 | 2.545 |

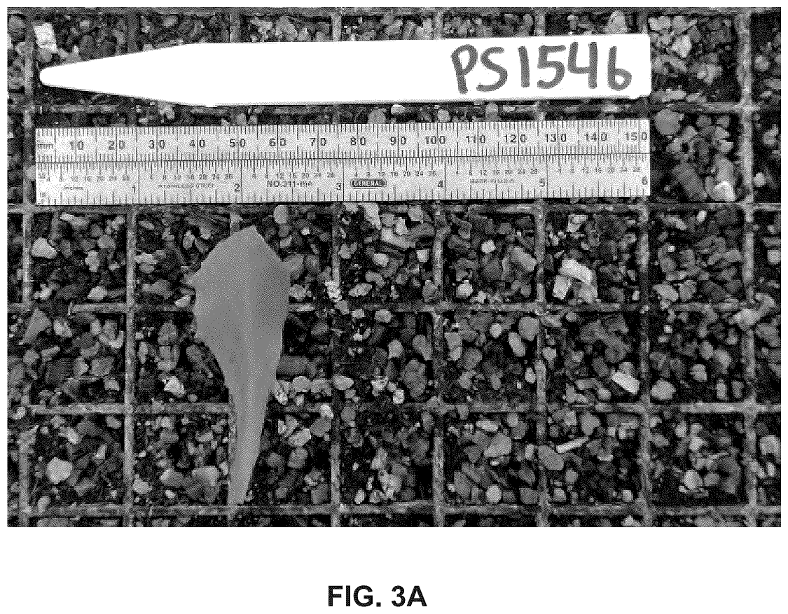

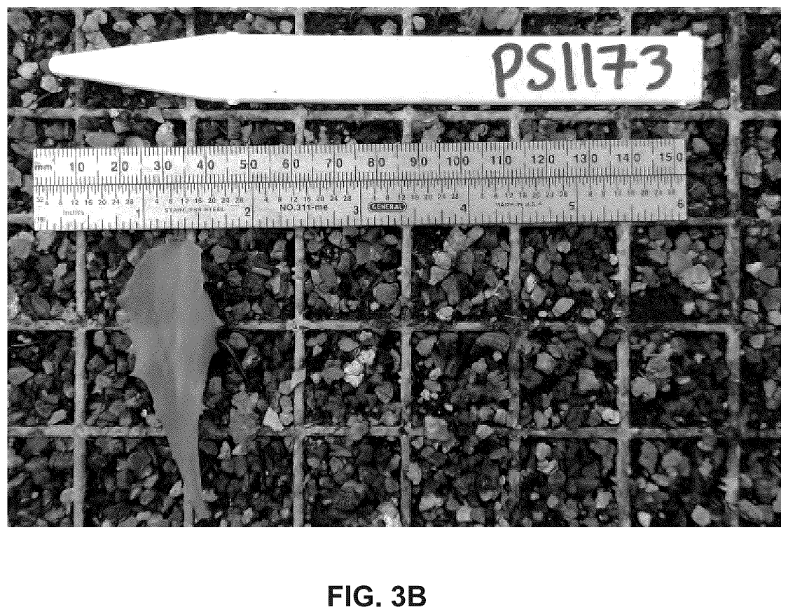

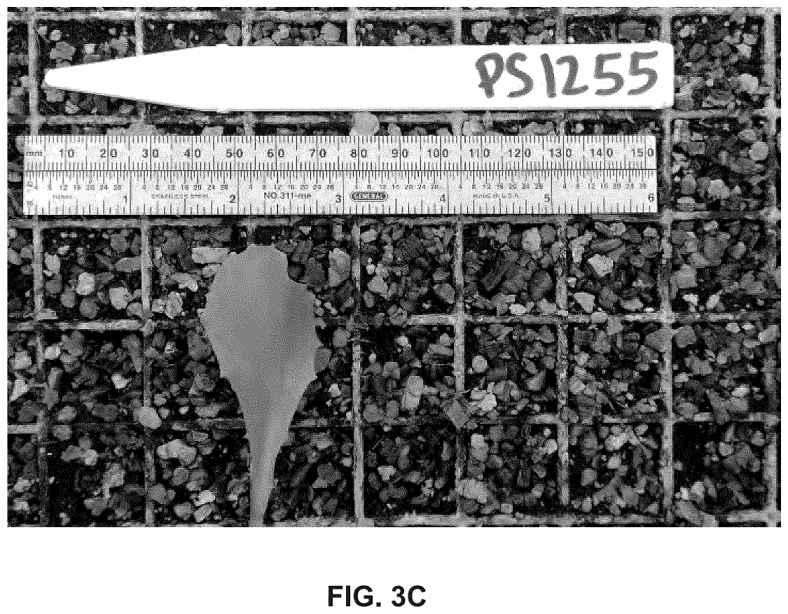

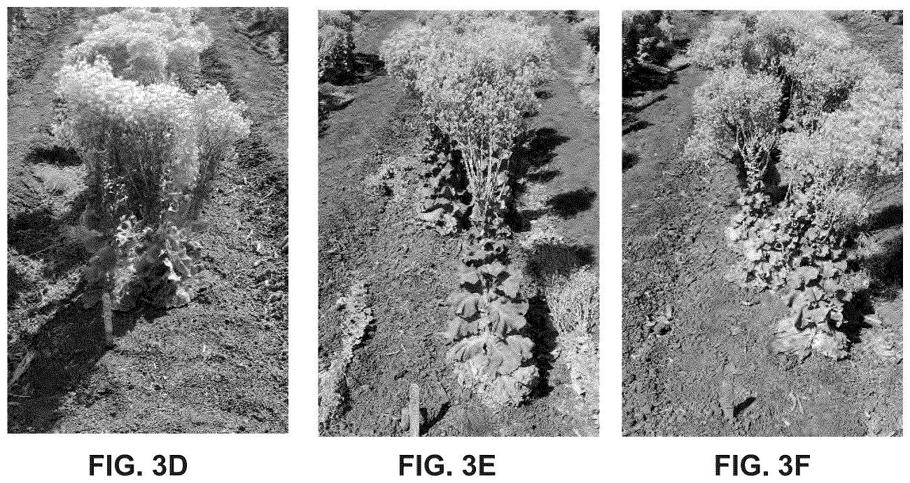

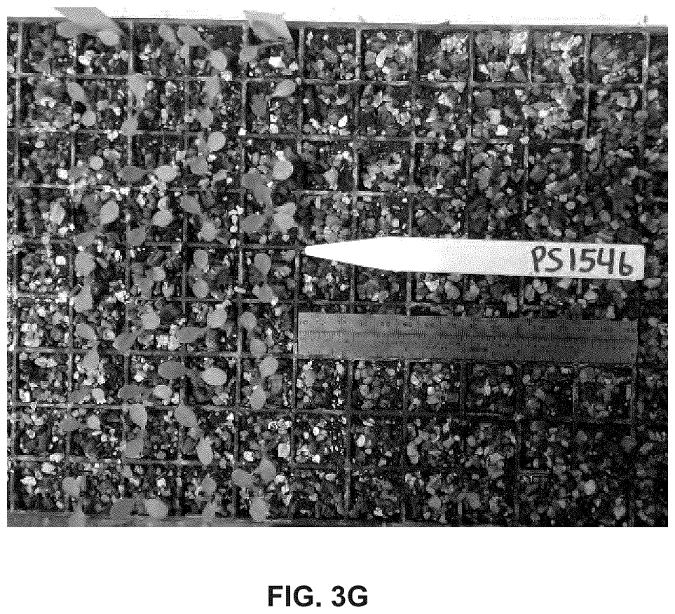

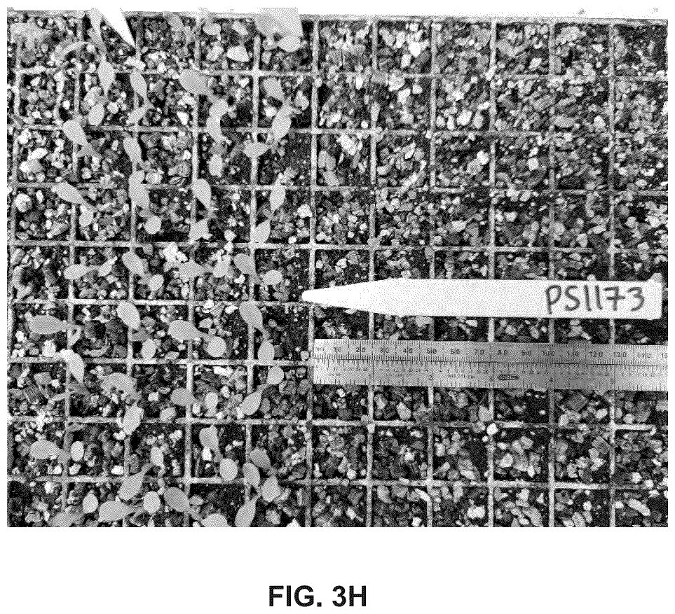

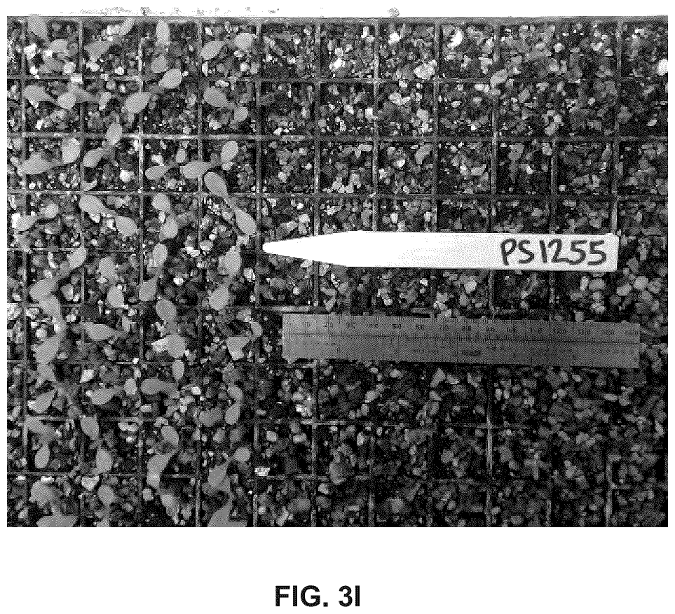

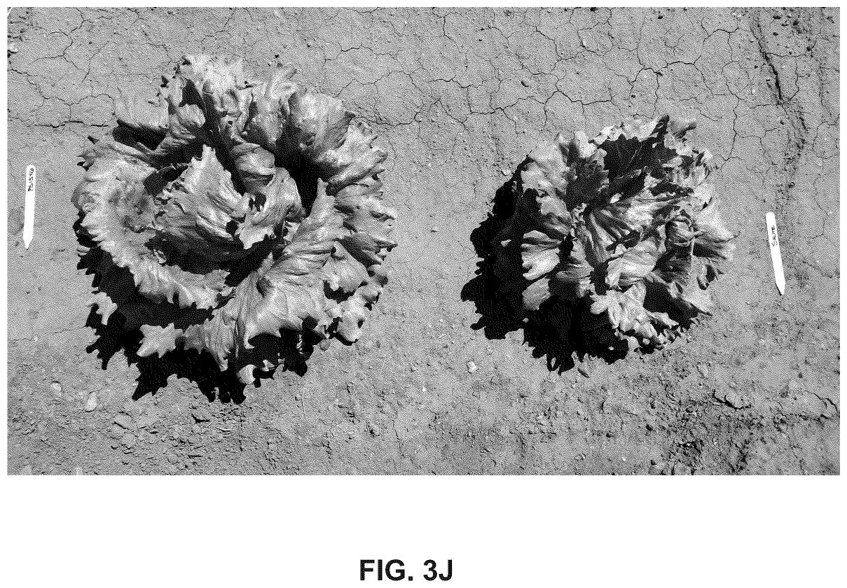

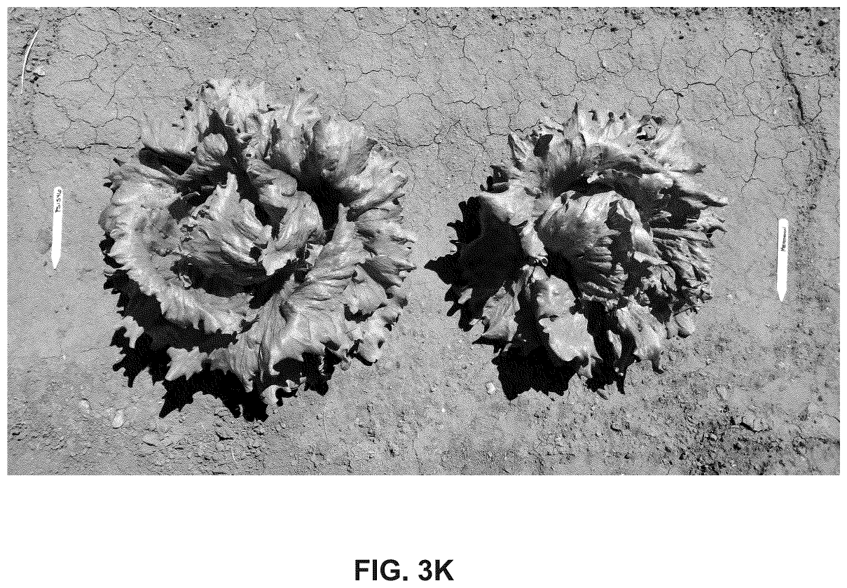

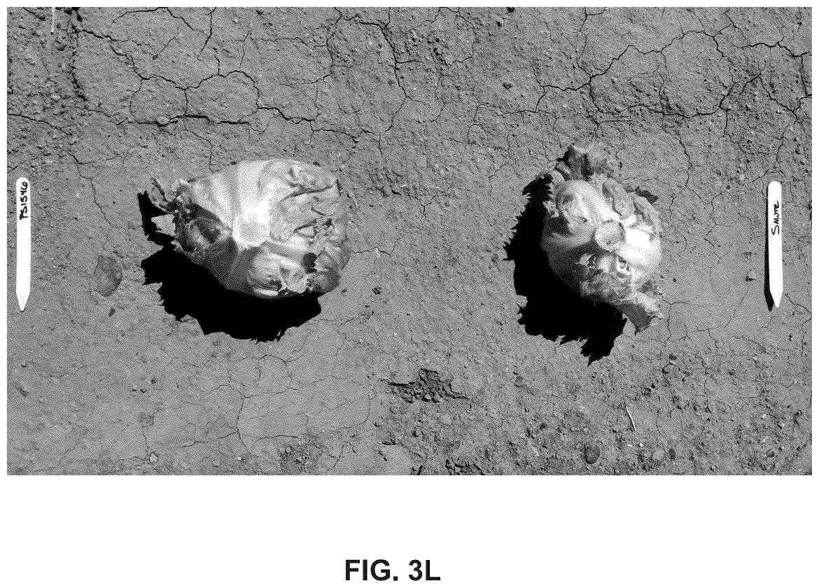

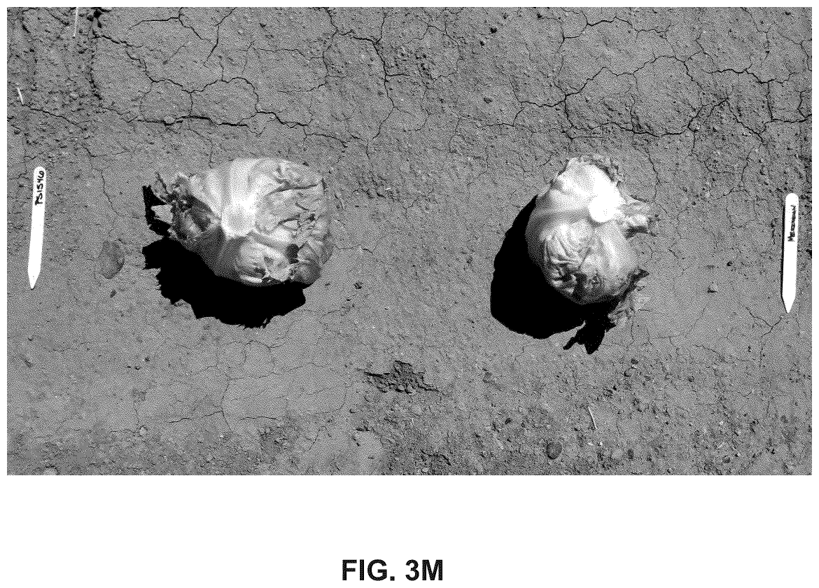

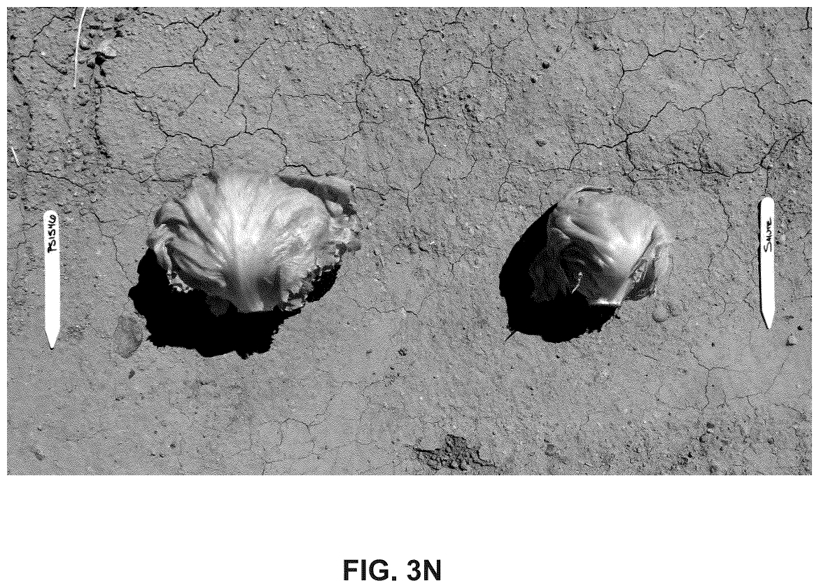

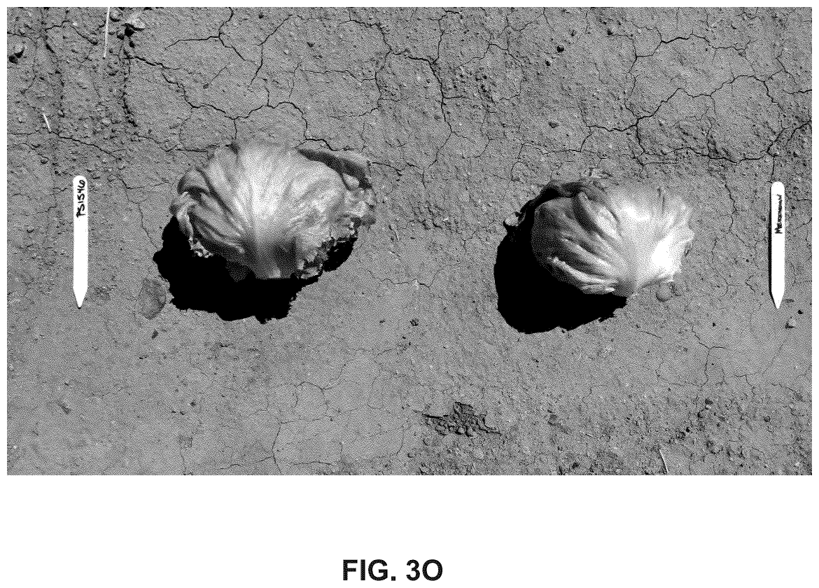

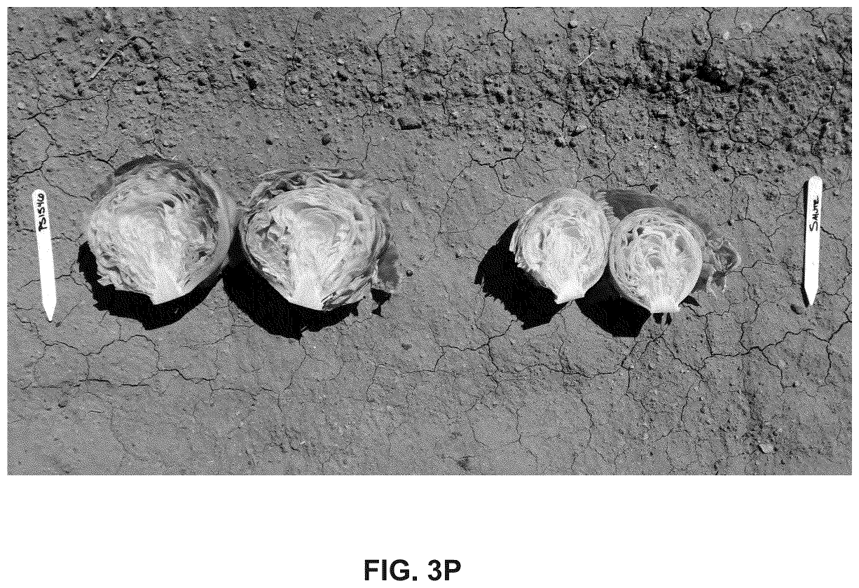

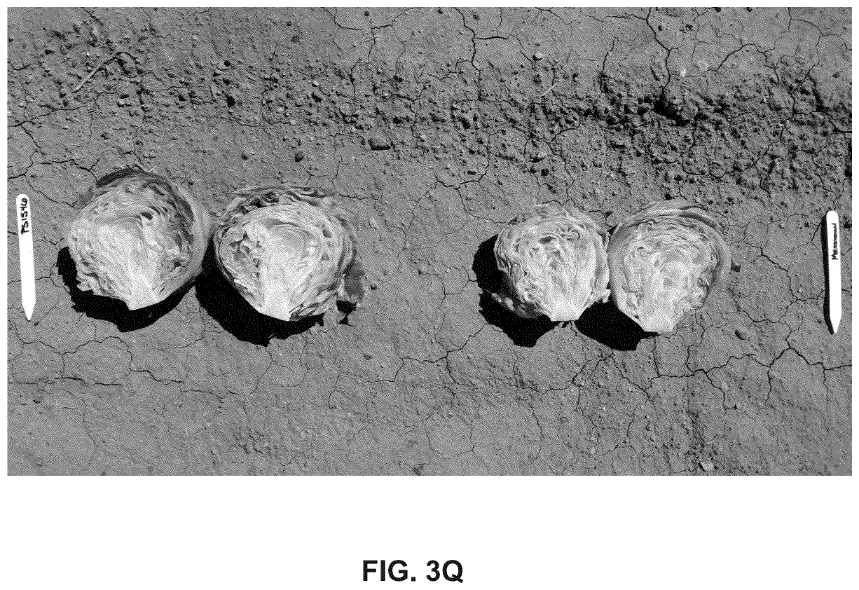

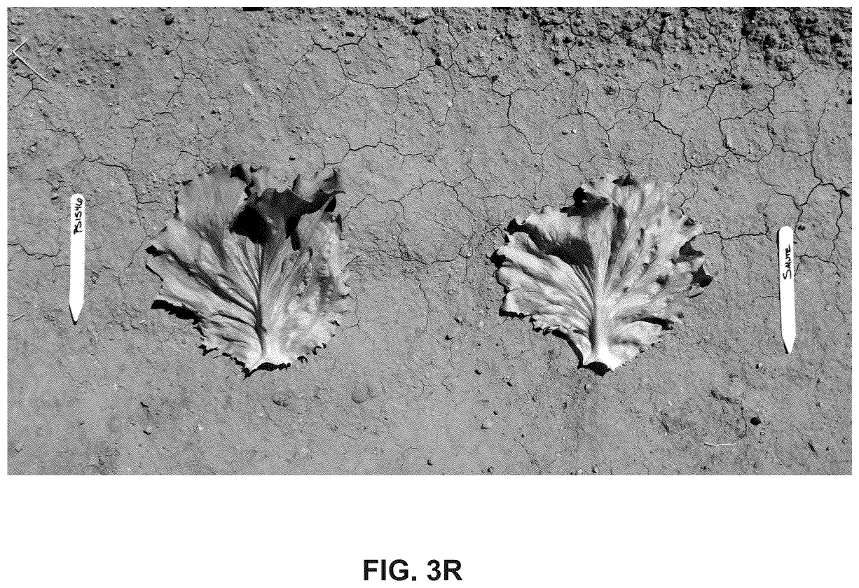

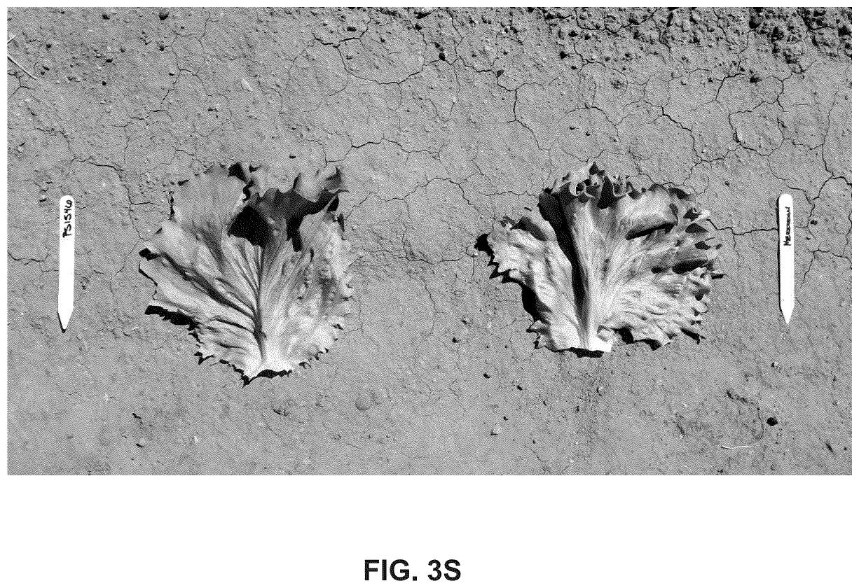

Further distinguishing features are apparent from the comparison of the variety ‘PS 1546’ with the varieties ‘Meridian’ and ‘Salute’ depicted in FIGS. 3A-3S.

### Objective Description of the Variety ‘PS 1902’

‘PS 1902’ is an inbred cutting/leaf lettuce variety. This variety is distinct and unique to all other cutting/leaf lettuce varieties due to its Fusarium resistance, TBSV resistance, and INSV resistance. Moreover, ‘PS 1902’ is a durable green leaf variety that has multiple resistances and still performs like a standard green leaf variety. In the U.S., green leaf is a rapidly growing market segment, having increased 68% over the last five years (USDA, 2002). When compared to reference varieties, ‘PS 1902’ has a larger frame length, increased weight, and larger heart length. ‘PS 1902’ is adapted to growing in regions such as the Southwest and West Coast regions of the United States, such as California and the Arizona desert. Additionally, ‘PS 1902’ is suitable for growing in the fall, winter, and spring for areas such as Yuma, Arizona, USA; and in the spring, summer, and fall for areas such as Salinas, California, USA. Lettuce variety ‘PS 1902’ is the result of numerous generations of plant selections chosen for its Fusarium resistance, TBSV resistance, and INSV resistance.

The variety has shown uniformity and stability for the traits, within the limits of environmental influence for the traits. It has been self-pollinated a sufficient number of generations with careful attention to uniformity of plant type. The line has been increased with continued observation for uniformity. No variant traits have been observed or are expected in variety ‘PS 1902’.
Lettuce variety ‘PS 1902’ has the following morphologic and other characteristics:
Plant type: Cutting/Leaf
Seed:
Color: Black (Grey brown)
Light dormancy: Light not required
Heat dormancy: Susceptible
Cotyledon to Fourth Leaf Stage:
Shape of cotyledons: Intermediate
Cotyledon length: 12.9 mm
Cotyledon width: 7.8 mm
Cotyledon index (length/width×10): 16.5
Shape of fourth leaf: Oval
Fourth leaf length: 28.1 mm
Fourth leaf width: 54.2 mm
Fourth leaf index (length/width×10): 5.2
Apical margin: Crenate/gnawed
Basal margin: Crenate/gnawed
Green color: Dark green
Anthocyanin distribution: Absent
Cupping: Slight
Reflexing: None
Mature Leaves:
Margin:
Incision depth (deepest penetration of the margin): Moderate
Indentation (finest divisions of the margin): Shallow dentate
Undulation of apical margin: Moderate
Color (Munsell): 5GY 4/8 (Dark green)
Anthocyanin distribution: Absent
Glossiness: Moderate
Blistering: Moderate
Thickness: Thick
Trichomes: Absent (smooth)
Plant:
Weight: 362.2 g
Spread of frame leaves: 39.3 cm
Heart Length (market trimmed with single cap leaf): 18.4 cm
Head Shape: Non-heading
Head size class: Medium
Head firmness: Loose
Butt:
Shape: Flat
Midrib: Moderately Raised
Plant Core:
Diameter at base of heart: 29.9 mm
Ratio of heart length/core diameter: 6.2
Core height from base to heart to apex: 41.5 mm
Bolting:
Number of days from first water to seed stalk emergence under summer conditions: 69
Bolting class: Slow
Mature seed stalk height: 96.8 cm
Mature seed stalk spread: 27.3 cm
Bolter leaves: Curved
Margin: Entire
Bolter habit:
Terminal inflorescence: Present
Lateral shoots: Present
Basal side shoots: Absent
Disease/Pest Resistance:
Downy Mildew: Intermediate resistance
Fusarium wilt Race 1: Resistant
TBSV: Resistant
INSV: Resistant
Stress Resistance:
Tipburn: High tip burn resistance

### Comparisons to Commercial Lettuce Variety

Table 19 below compares characteristics of lettuce variety ‘PS 1902’ with the lettuce varieties ‘Big Star’ (U.S. Plant Variety Protection Certificate 200200013) and ‘Bergam's Green’ (U.S. Plant Variety Protection Certificate 200500262). Column 1 lists the characteristics, column 2 shows the characteristics for lettuce variety ‘PS 1902’, column 3 shows the characteristics for lettuce variety ‘Big Star’, and column 4 shows the characteristics for lettuce variety ‘Bergam's Green’.

TABLE 19

| Characteristic | ‘PS 1902’ | | ‘Big Star’ | | ‘Bergam's Green’ | |
|---|---|---|---|---|---|---|
| Seed Color | Black (Grey | | White (Silver | | Black (Grey | |
| | Brown) | | Gray) | | Brown) | |
| Seed | Light not | | Light required | | Light not | |
| | required | | | | required | |
| Light dormancy | | | | | | |
| Frame Length | 39.3 | cm | 36.9 | cm | 36.4 | cm |
| Weight | 362.2 | g | 297.9 | g | 361.1 | g |
| Heart Length | 18.4 | mm | 16.6 | mm | 16.8 | mm |
| Core Diameter | 29.9 | mm | 26.2 | mm | 30.2 | mm |
| Core Length | 41.5 | mm | 38.2 | mm | 35.9 | mm |

| | | | |
|---|---|---|---|
| Color (Munsell) | 5GY 4/8 | 5GY 4/6 | 5GY 4/4 |

Tables 20A-20C below show results of a first trial that compares the heart weight, heart length, core length, core diameter, and frame diameter of 20 plants of the lettuce variety ‘PS 1902’ (Table 20A) with those of 20 plants of lettuce variety ‘Big Star’ (Table 20B) and 20 plants of lettuce variety ‘Bergam's Green’ (Table 20C). The heart weights shown are total heart weights.

TABLE 20A

| | | Heart | Core | Core | Frame |
|---|---|---|---|---|---|
| | Heart Wt. | Length | Length | Diameter | Diameter |
| ‘PS 1902’ | (g) | (cm) | (mm) | (mm) | (cm) |
| Max | 418 | 14 | 50.8 | 31.9 | 49.4 |
| Min | 250 | 7.4 | 22.4 | 23.8 | 36.1 |
| Average | 332.9 | 10.1 | 36.5 | 27.1 | 39.3 |
| Std. Dev. | 51.35 | 1.698 | 6.423 | 2.419 | 2.806 |

TABLE 20B

| | | Heart | Core | Core | Frame |
|---|---|---|---|---|---|
| | Heart Wt. | Length | Length | Diameter | Diameter |
| ‘Big Star’ | (g) | (cm) | (mm) | (mm) | (cm) |
| Max | 370 | 9.5 | 40.1 | 30.3 | 36.1 |
| Min | 173 | 6.7 | 27.1 | 21.6 | 31.8 |
| Average | 278.0 | 8.7 | 33.7 | 25.3 | 34.1 |
| Std. Dev. | 51.41 | 0.7499 | 3.217 | 2.352 | 1.363 |

TABLE 20C

| | | Heart | Core | Core | Frame |
|---|---|---|---|---|---|
| ‘Bergam's | Heart Wt. | Length | Length | Diameter | Diameter |
| Green’ | (g) | (cm) | (mm) | (mm) | (cm) |
| Max | 429 | 14 | 36.7 | 30.3 | 36.1 |
| Min | 278 | 9 | 23.4 | 26.1 | 31.6 |
| Average | 334.6 | 11.5 | 30.8 | 27.7 | 33.5 |
| Std. Dev. | 41.35 | 1.525 | 4.098 | 1.211 | 1.106 |

Tables 21A-21C below show results of a second trial that compares the heart weight, heart length, core length, core diameter, and frame diameter of 20 plants of the lettuce variety

‘PS 1902’ (Table 21A) with those of 20 plants of lettuce variety ‘Big Star’ (Table 21B) and 20 plants of lettuce variety ‘Bergam's Green’ (Table 21C). The heart weights shown are total heart weights.

TABLE 21A

| | | Heart | Core | Core | Frame |
|---|---|---|---|---|---|
| | Heart Wt. | Length | Length | Diameter | Diameter |
| ‘PS 1902’ | (g) | (cm) | (mm) | (mm) | (cm) |
| Max | 533 | 25.4 | 42.2 | 41.6 | 42.4 |
| Min | 243 | 14.3 | 20.8 | 25 | 34.3 |
| Average | 382.9 | 19.6 | 34.2 | 33.4 | 38.4 |
| Std. Dev. | 68.38 | 2.535 | 5.162 | 4.115 | 2.454 |

TABLE 21B

| | | Heart | Core | Core | Frame |
|---|---|---|---|---|---|
| | Heart Wt. | Length | Length | Diameter | Diameter |
| ‘Big Star’ | (g) | (cm) | (mm) | (mm) | (cm) |
| Max | 362 | 18 | 38.7 | 30.2 | 42.3 |
| Min | 205 | 12 | 22.1 | 23.7 | 31.5 |
| Average | 280.8 | 15.2 | 29.2 | 25.9 | 37.1 |
| Std. Dev. | 48.09 | 1.771 | 4.592 | 1.651 | 2.842 |

TABLE 21C

| | | Heart | Core | Core | Frame |
|---|---|---|---|---|---|
| ‘Bergam's | Heart Wt. | Length | Length | Diameter | Diameter |
| Green’ | (g) | (cm) | (mm) | (mm) | (cm) |
| Max | 427 | 18.4 | 34.3 | 33.3 | 41.4 |
| Min | 209 | 12.5 | 20.3 | 23.5 | 32.9 |
| Average | 331.8 | 16.1 | 29.9 | 29.2 | 38.0 |
| Std. Dev. | 53.52 | 1.552 | 3.230 | 2.675 | 2.494 |

Tables 22A-22C below show results of a third trial that compares the heart weight, heart length, core length, core diameter, and frame diameter of 20 plants of the lettuce variety ‘PS 1902’ (Table 22A) with those of 20 plants of lettuce variety ‘Big Star’ (Table 22B) and 20 plants of lettuce variety ‘Bergam's Green’ (Table 22C). The heart weights shown are total heart weights.

TABLE 22A

| | | Heart | Core | Core | Frame |
|---|---|---|---|---|---|
| | Heart Wt. | Length | Length | Diameter | Diameter |
| ‘PS 1902’ | (g) | (cm) | (mm) | (mm) | (cm) |
| Max | 350 | 26.9 | 58 | 32 | 47.1 |
| Min | 233 | 22.1 | 38 | 26 | 39.6 |
| Average | 302.0 | 23.9 | 49.0 | 29.1 | 42.3 |
| Std. Dev. | 33.85 | 1.656 | 5.256 | 1.744 | 2.265 |

TABLE 22B

| | | Heart | Core | Core | Frame |
|---|---|---|---|---|---|
| | Heart Wt. | Length | Length | Diameter | Diameter |
| ‘Big Star’ | (g) | (cm) | (mm) | (mm) | (cm) |
| Max | 339 | 27.1 | 54 | 31 | 44.8 |
| Min | 215 | 19.7 | 38 | 22 | 41.2 |
| Average | 275.8 | 24.1 | 47.5 | 28.3 | 42.8 |
| Std. Dev. | 31.74 | 1.806 | 4.161 | 2.314 | 0.9904 |

TABLE 22C

| | | Heart | Core | Core | Frame |
|---|---|---|---|---|---|
| ‘Bergam's | Heart Wt. | Length | Length | Diameter | Diameter |
| Green’ | (g) | (cm) | (mm) | (mm) | (cm) |
| Max | 422 | 25.1 | 53 | 37 | 44.3 |
| Min | 308 | 19.8 | 35 | 31 | 39.4 |
| Average | 359.1 | 22.8 | 45.2 | 34.0 | 41.1 |
| Std. Dev. | 34.15 | 1.647 | 5.382 | 1.605 | 1.279 |

Tables 23A-23C below show results of a fourth trial that compares the heart weight, heart length, core length, core diameter, and frame diameter of 20 plants of the lettuce variety ‘PS 1902’ (Table 23A) with those of 20 plants of lettuce variety ‘Big Star’ (Table 23B) and 20 plants of lettuce variety ‘Bergam's Green’ (Table 23C). The heart weights shown are total heart weights.

TABLE 23A

| | | Heart | Core | Core | Frame |
|---|---|---|---|---|---|
| | Heart Wt. | Length | Length | Diameter | Diameter |
| ‘PS 1902’ | (g) | (cm) | (mm) | (mm) | (cm) |
| Max | 632 | 23.2 | 57 | 37 | 46.4 |
| Min | 322 | 17.7 | 34 | 21 | 30.4 |
| Average | 431.2 | 20.0 | 46.3 | 30.1 | 37.4 |
| Std. Dev. | 72.35 | 1.776 | 5.476 | 3.161 | 4.615 |

TABLE 23B

| | | Heart | Core | Core | Frame |
|---|---|---|---|---|---|
| | Heart Wt. | Length | Length | Diameter | Diameter |
| ‘Big Star’ | (g) | (cm) | (mm) | (mm) | (cm) |
| Max | 408 | 20.5 | 49 | 29 | 37.2 |
| Min | 296 | 16 | 34 | 20 | 31.2 |
| Average | 357.3 | 18.5 | 42.4 | 25.2 | 33.8 |
| Std. Dev. | 34.85 | 1.276 | 4.558 | 2.215 | 1.672 |

TABLE 23C

| | | Heart | Core | Core | Frame |
|---|---|---|---|---|---|
| ‘Bergam's | Heart Wt. | Length | Length | Diameter | Diameter |
| Green’ | (g) | (cm) | (mm) | (mm) | (cm) |
| Max | 528 | 19.8 | 49 | 33 | 38.9 |
| Min | 327 | 15.2 | 32 | 27 | 30.4 |
| Average | 418.9 | 16.8 | 38.1 | 30.1 | 33.0 |
| Std. Dev. | 60.39 | 1.200 | 4.576 | 1.849 | 2.084 |

Tables 24A-24C below compares the fourth leaf width, fourth leaf length, cotyledon leaf width, cotyledon leaf length, stalk length, and frame of 20 plants of the lettuce variety ‘PS 1902’ (Table 24A) with those of 20 plants of lettuce variety ‘Big Star’ (Table 24B) and 20 plants of lettuce variety ‘Bergam's Green’ (Table 24C). The heart weights shown are total heart weights.

TABLE 24A

| | Fourth | Fourth | Cot. | | | |
|---|---|---|---|---|---|---|
| | leaf | leaf | leaf | Cot. leaf | | |
| | width | length | width | length | Stalk | Frame |
| ‘PS 1902’ | (mm) | (mm) | (mm) | (mm) | (cm) | (cm) |
| Max | 62 | 33 | 8 | 14 | 115.8 | 30.2 |
| Min | 46 | 23 | 7 | 11 | 80.1 | 24.3 |
| Average | 54.2 | 28.1 | 7.8 | 12.9 | 96.8 | 27.3 |
| Std. Dev. | 4.360 | 3.086 | 0.4443 | 0.9333 | 8.375 | 1.842 |

TABLE 24B

| | Fourth | Fourth | Cot. | | | |
|---|---|---|---|---|---|---|
| | leaf | leaf | leaf | Cot. leaf | | |
| | width | length | width | length | Stalk | Frame |
| ‘Big Star’ | (mm) | (mm) | (mm) | (mm) | (cm) | (cm) |
| Max | 68 | 60 | 8 | 14 | 106.7 | 31.3 |
| Min | 48 | 27 | 6 | 8 | 88.1 | 20.3 |
| Average | 58.8 | 31.9 | 7 | 11.2 | 97.9 | 24.3 |
| Std. Dev. | 5.486 | 6.905 | 0.5620 | 1.531 | 4.468 | 2.905 |

TABLE 24C

| | Fourth | Fourth | Cot. | | | |
|---|---|---|---|---|---|---|
| | leaf | leaf | leaf | Cot. leaf | | |
| ‘Bergam's | width | length | width | length | Stalk | Frame |
| Green’ | (mm) | (mm) | (mm) | (mm) | (cm) | (cm) |
| Max | 56 | 32 | 9 | 14 | 110.5 | 27.4 |
| Min | 40 | 22 | 7 | 9 | 89.5 | 21.5 |
| Average | 47.2 | 27.9 | 7.8 | 11.6 | 97.0 | 24.9 |
| Std. Dev. | 4.120 | 2.700 | 0.5501 | 1.356 | 5.570 | 1.388 |

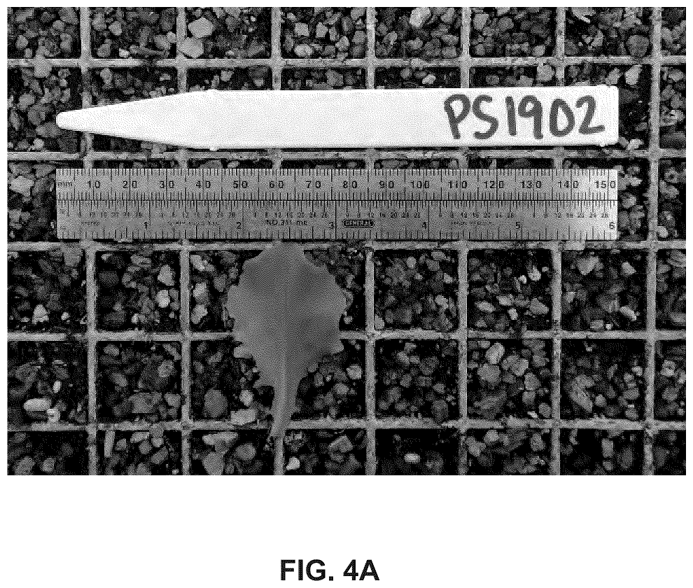

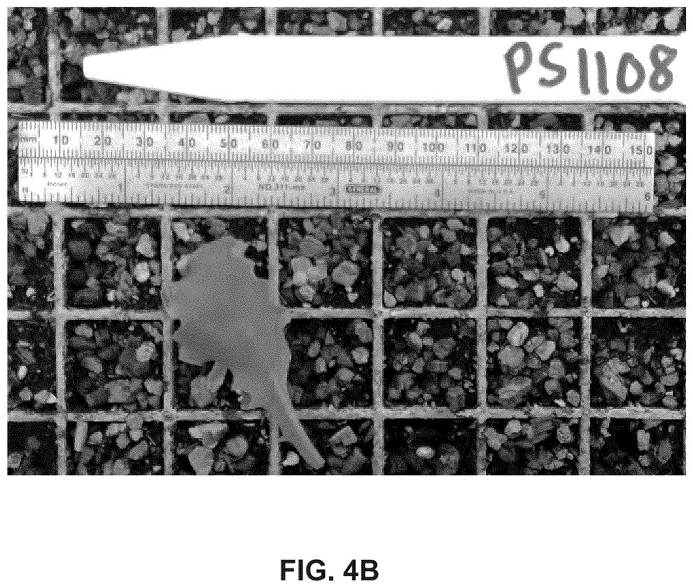

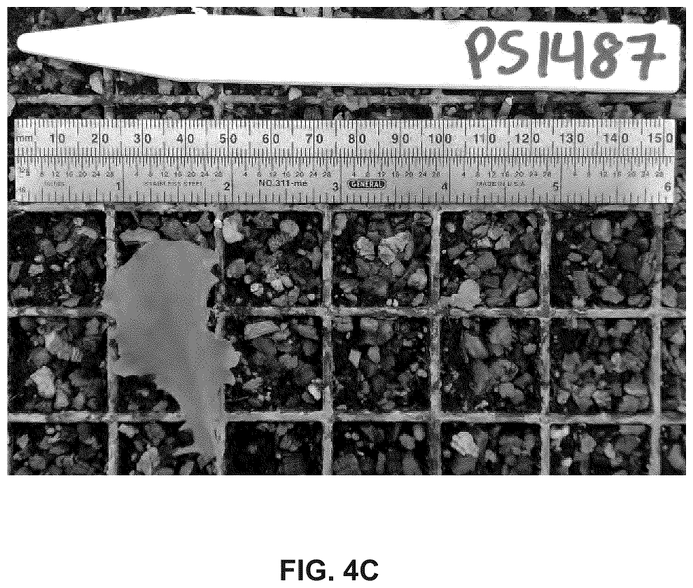

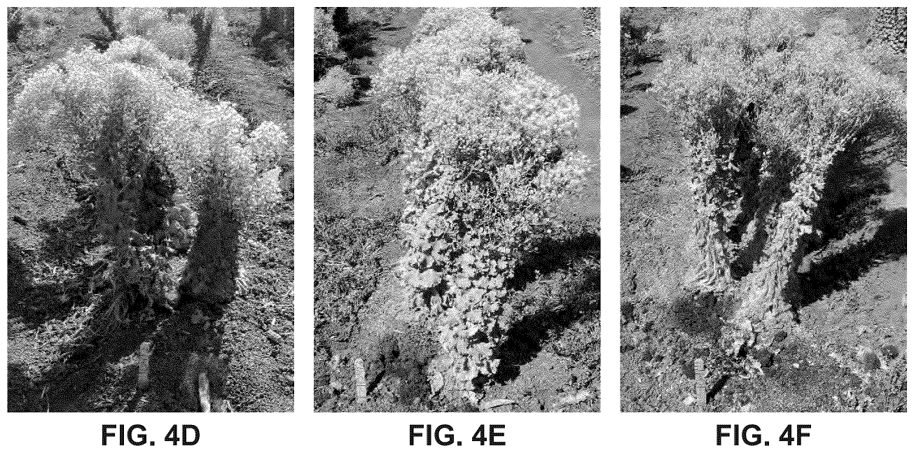

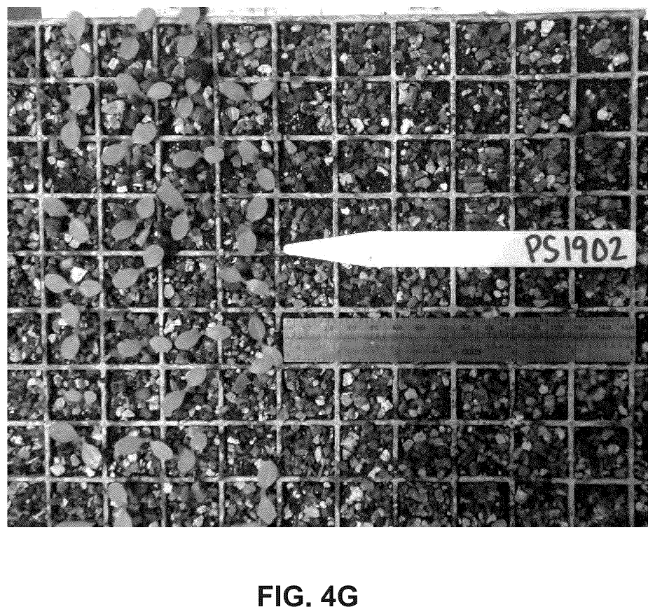

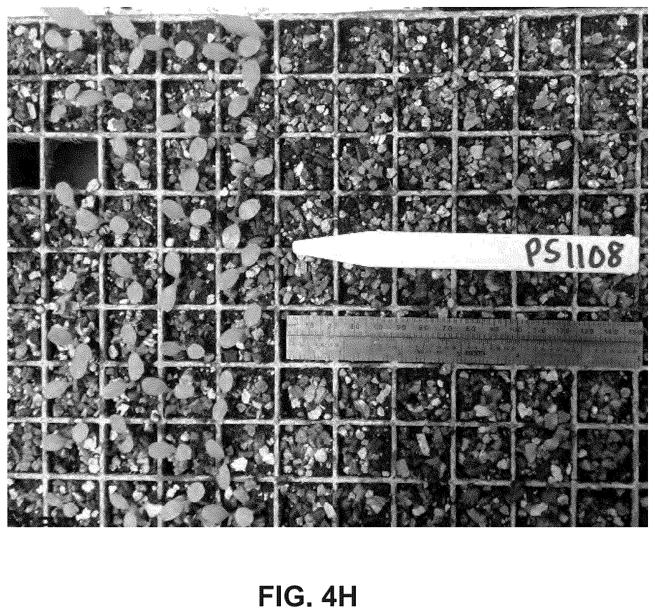

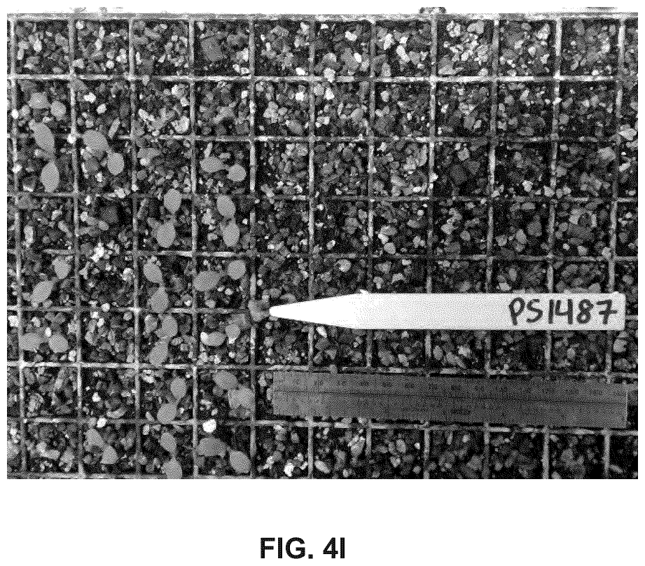

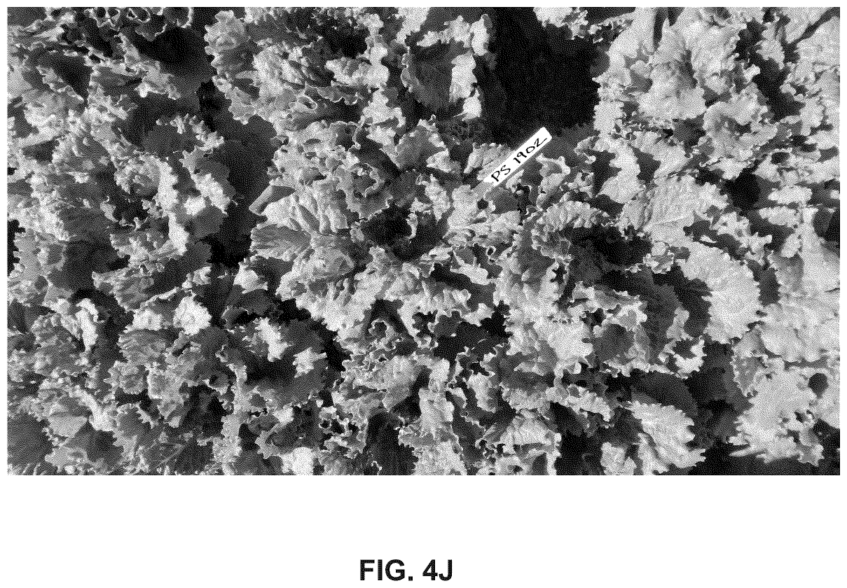

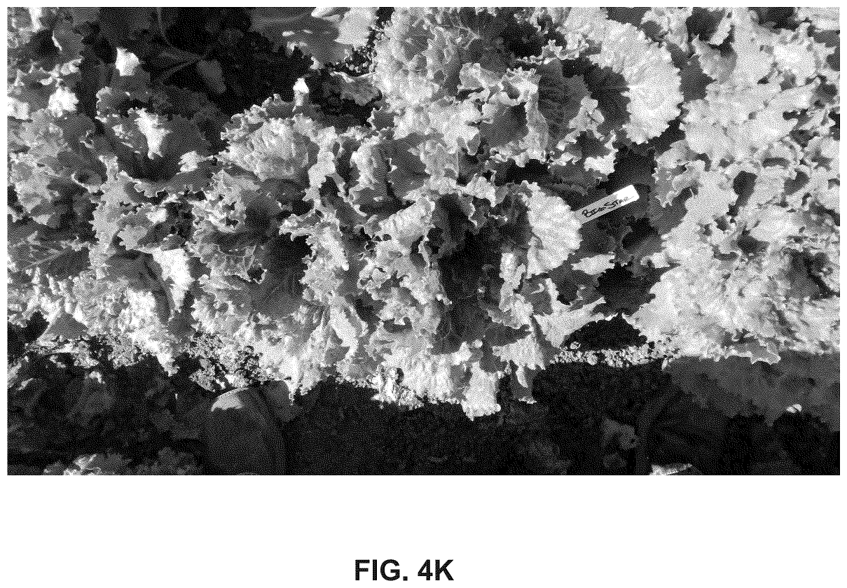

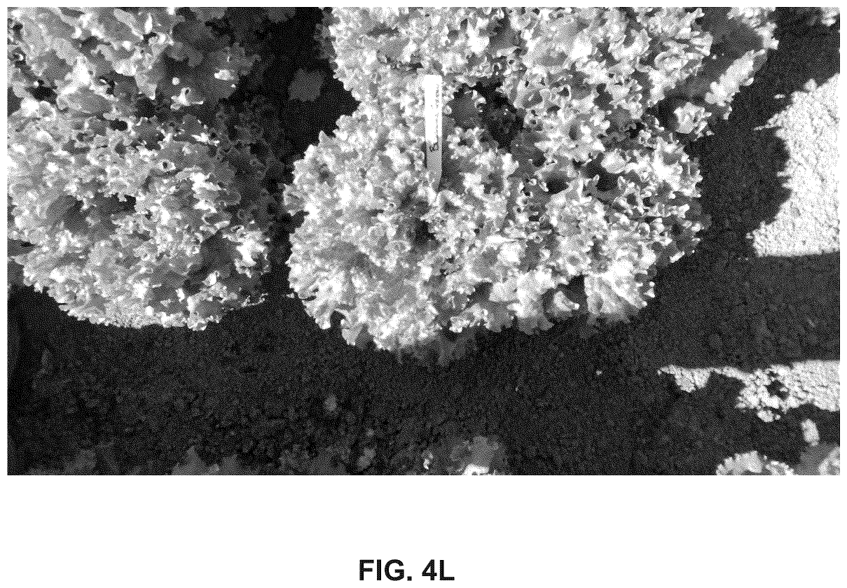

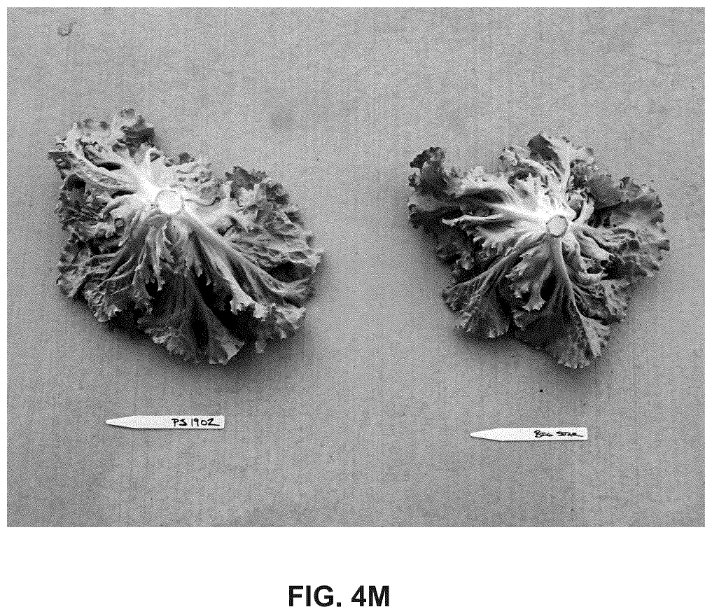

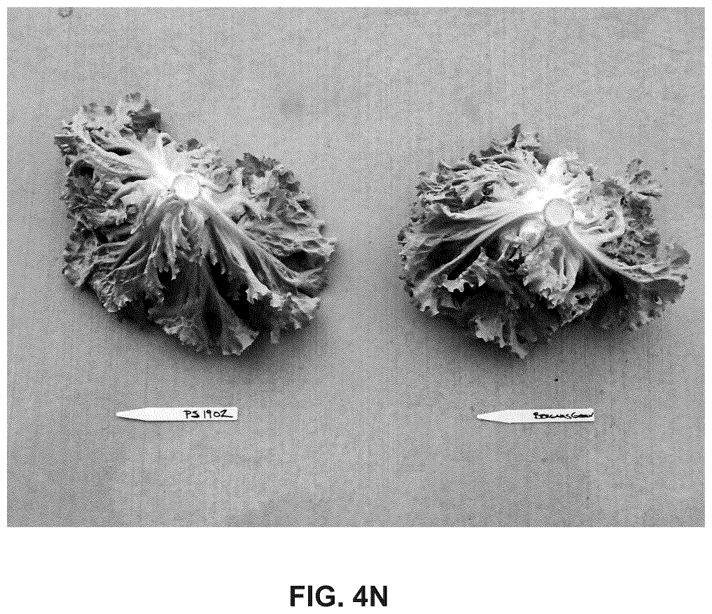

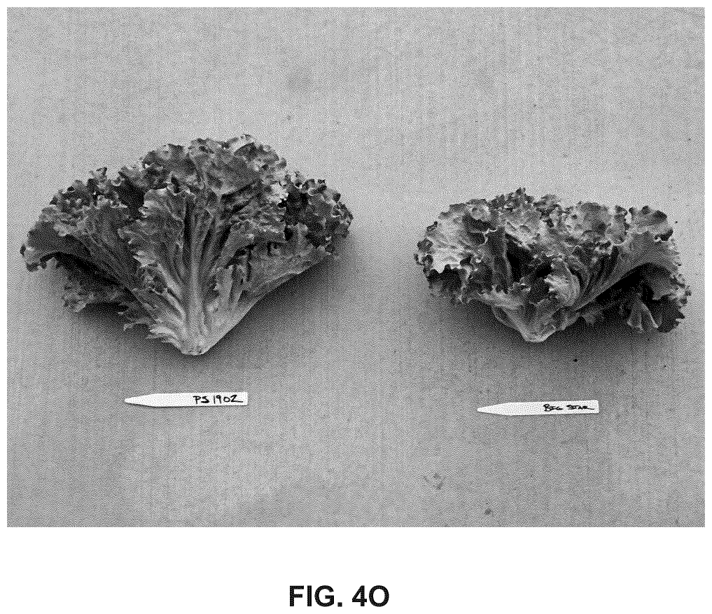

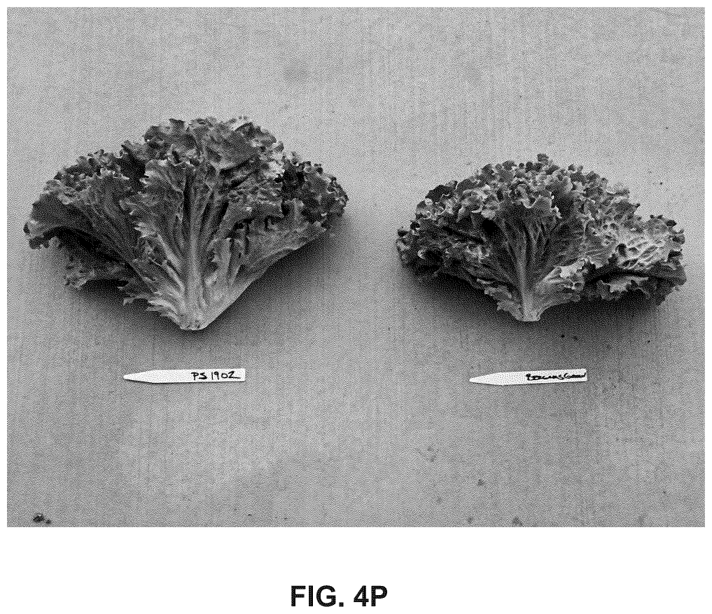

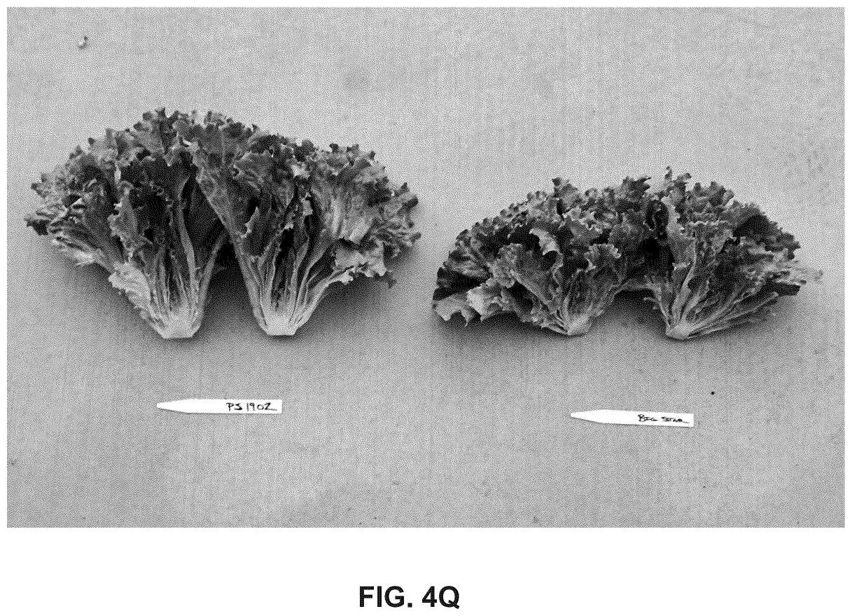

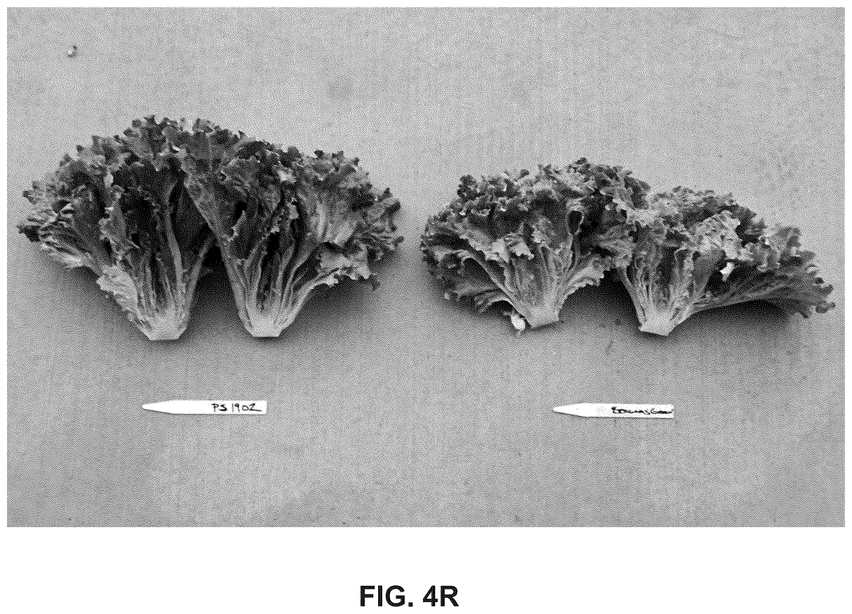

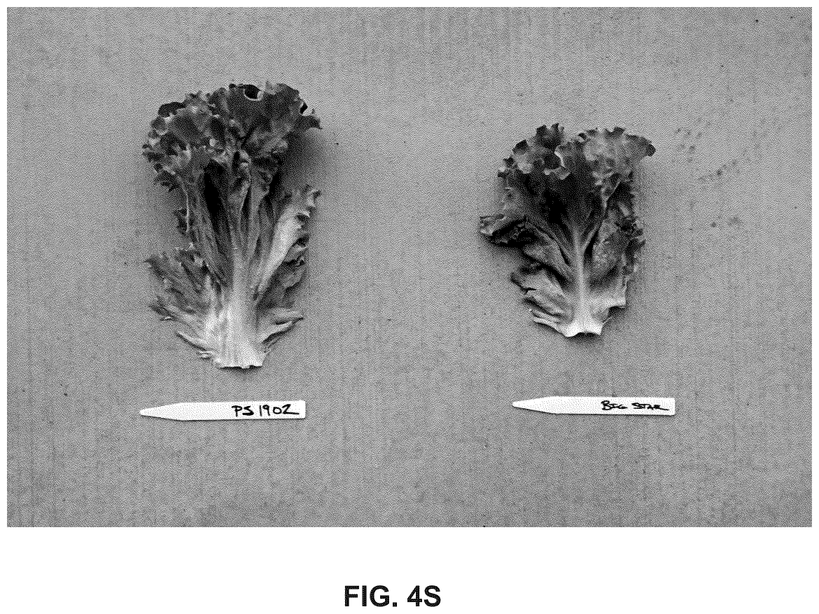

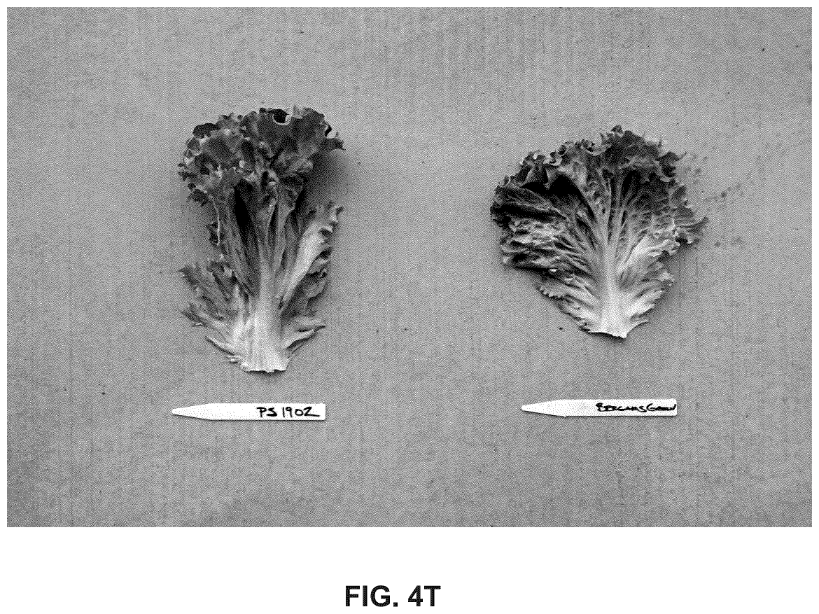

Further distinguishing features are apparent from the comparison of the variety ‘PS 1902’ with the varieties ‘Big Star’ and ‘Bergam's Green’ depicted in FIGS. 4A-4T.

### Further Embodiments

### Breeding

In lettuce breeding, lines are selected for their appropriate characteristics. To optimize crossing, it is important to note that lettuce is an obligate self-pollinating species. This means that the pollen is shed before stigma emergence, assuring 100% self-fertilization. Since each lettuce flower is an aggregate of about 10-20 individual florets (typical of the Compositae family), manual removal of the anther tubes containing the pollen is performed by procedures well known in the art of lettuce breeding.

The manual removal of anther tubes, though an effective means to ensure the removal of all self pollinating possibilities, is very tedious and time consuming when a large number of crosses are to be made. The breeders have therefore adapted a well documented and modified method of making crosses more efficiently using these methods. This particular cross was made by first misting the designated male flowers to wash the pollen off prior to fertilization. This process of misting is a proven and effective means of pollen removal that assures crossing or hybridization. About 60-90 minutes past sunrise, flowers to be used for crossings are selected. The basis for selection are open flowers, with the stigma emerged and the pollen visibly attached to the single stigma (about 10-20 stigma). Using 3-4 pumps of water from a regular spray bottle, the pollen is washed off with enough pressure to dislodge the pollen grains, but not enough to damage the style. Excess water is dried off with clean paper towels. About 30 minutes later, the styles should spring back up and the two lobes of the stigma are visibly open in a “V” shape. Pollen from another variety or donor parent is then introduced by gently rubbing the stigma and style of the donor parent to the maternal parent. Tags with the pertinent information on date and pedigree are then secured to the flowers in order to keep track.

About 2-3 weeks after pollination, seeds are harvested when the involucre have matured. The seeds are eventually sown and in the presence of markers such as leaf color or leaf margins, the selfed or maternal seedlings or plants are identified. Generally, there are no visible markers and breeders must wait until the F2 generations when expected segregation patterns for the genetic character of interest can be followed. This latter situation mandates a lengthy wait to determine if hybrids are produced. Two relevant references teaching methods for out crossing lettuce are: (1) Ryder, E. J. and A. S. Johnson. 1974. Mist depollination of lettuce flowers. Hortscience 9:584; and (2) Nagata, R. T. 1992. Clip and Wash Method of Emasculation for Lettuce. Hortscience 27 (8): 907-908 both of which are hereby incorporated by reference in their entirety for the purpose of providing details on the techniques well known in the art.

### Selection

In addition to crossing, selection may be used to identify and isolate new lettuce lines. In lettuce selection, lettuce seeds are planted, the plants are grown and single plant selections are made of plants with desired characteristics. Such characteristics may include improved head and frame size, deeper or darker green leaf color, etc. Seed from the single plant selections are harvested, separated from seeds of the other plants in the field and re-planted. The plants from the selected seed are monitored to determine if they exhibit the desired characteristics of the originally selected line. Selection work is continued over multiple generations to increase the uniformity of the new line.

### Deposit Information

### Lettuce Variety ‘PS 1540’

A deposit of at least 625 seeds of the lettuce variety ‘PS 1540’ was made with the American Type Culture Collection (ATCC), ATCC Patent Depository, 10801 University Boulevard, Manassas, Virginia, 20110, USA, and assigned ATCC number X1. The seeds deposited with the ATCC on DATE were obtained from the seed of the variety maintained by Pinnacle Seed, Inc., P.O. Box 222672, Carmel, California 93923, United States of America since prior to the filing date of the application. Access to this deposit will be available during the pendency of this application to persons determined by the Commissioner of Patents and Trademarks to be entitled thereto under 37 C.F.R. § 1.14 and 35 U.S.C. § 122. Upon issuance, the Applicant will make the deposit available to the public consistent with all of the requirements of 37 C.F.R. § 1.801-1.809. This deposit of the lettuce variety ‘PS 1540’ will be maintained in the ATCC, which is a public depository, for a period of 30 years, or at least 5 years after the most recent request for a sample of the deposit, or for the effective life of the patent, whichever is longer, and will be replaced if it becomes nonviable during that period. Applicant has no authority to waive any restrictions imposed by law on the transfer of biological material or its transportation in commerce. Applicant does not waive any infringement of rights granted under this patent or under the Plant Variety Protection Act (7 USC 2321 et seq.).

### Lettuce Variety ‘PS 1541’

A deposit of at least 625 seeds of the lettuce variety ‘PS 1541’ was made with the American Type Culture Collection (ATCC), ATCC Patent Depository, 10801 University Boulevard, Manassas, Virginia, 20110, USA, and assigned ATCC number X2. The seeds deposited with the ATCC on DATE were obtained from the seed of the variety maintained by Pinnacle Seed, Inc., P.O. Box 222672, Carmel, California 93923, United States of America since prior to the filing date of the application. Access to this deposit will be available during the pendency of this application to persons determined by the Commissioner of Patents and Trademarks to be entitled thereto under 37 C.F.R. § 1.14 and 35 U.S.C. § 122. Upon issuance, the Applicant will make the deposit available to the public consistent with all of the requirements of 37 C.F.R. § 1.801-1.809. This deposit of the lettuce variety ‘PS 1541’ will be maintained in the ATCC, which is a public depository, for a period of 30 years, or at least 5 years after the most recent request for a sample of the deposit, or for the effective life of the patent, whichever is longer, and will be replaced if it becomes nonviable during that period. Applicant has no authority to waive any restrictions imposed by law on the transfer of biological material or its transportation in commerce. Applicant does not waive any infringement of rights granted under this patent or under the Plant Variety Protection Act (7 USC 2321 et seq.).

### Lettuce Variety ‘PS 1546’

A deposit of at least 625 seeds of the lettuce variety ‘PS 1546’ was made with the American Type Culture Collection (ATCC), ATCC Patent Depository, 10801 University Boulevard, Manassas, Virginia, 20110, USA, and assigned ATCC number X4. The seeds deposited with the ATCC on DATE were obtained from the seed of the variety maintained by Pinnacle Seed, Inc., P.O. Box 222672, Carmel, California 93923, United States of America since prior to the filing date of the application. Access to this deposit will be available during the pendency of this application to persons determined by the Commissioner of Patents and Trademarks to be entitled thereto under 37 C.F.R. § 1.14 and 35 U.S.C. § 122. Upon issuance, the Applicant will make the deposit available to the public consistent with all of the requirements of 37 C.F.R. § 1.801-1.809. This deposit of the lettuce variety ‘PS 1546’ will be maintained in the ATCC, which is a public depository, for a period of 30 years, or at least 5 years after the most recent request for a sample of the deposit, or for the effective life of the patent, whichever is longer, and will be replaced if it becomes nonviable during that period. Applicant has no authority to waive any restrictions imposed by law on the transfer of biological material or its transportation in commerce. Applicant does not waive any infringement of rights granted under this patent or under the Plant Variety Protection Act (7 USC 2321 et seq.).

### Lettuce Variety ‘PS 1902’

A deposit of at least 625 seeds of the lettuce variety ‘PS 1902’ was made with the American Type Culture Collection (ATCC), ATCC Patent Depository, 10801 University Boulevard, Manassas, Virginia, 20110, USA, and assigned ATCC number X11. The seeds deposited with the ATCC on DATE were obtained from the seed of the variety maintained by Pinnacle Seed, Inc., P.O. Box 222672, Carmel, California 93923, United States of America since prior to the filing date of the application. Access to this deposit will be available during the pendency of this application to persons determined by the Commissioner of Patents and Trademarks to be entitled thereto under 37 C.F.R. § 1.14 and 35 U.S.C. § 122. Upon issuance, the Applicant will make the deposit available to the public consistent with all of the requirements of 37 C.F.R. § 1.801-1.809. This deposit of the lettuce variety ‘PS 1902’ will be maintained in the ATCC, which is a public depository, for a period of 30 years, or at least 5 years after the most recent request for a sample of the deposit, or for the effective life of the patent, whichever is longer, and will be replaced if it becomes nonviable during that period. Applicant has no authority to waive any restrictions imposed by law on the transfer of biological material or its transportation in commerce. Applicant does not waive any infringement of rights granted under this patent or under the Plant Variety Protection Act (7 USC 2321 et seq.).

What is claimed:

1. A Lactuca sativa seed designated as ‘PS 1541’, representative sample of seed having been deposited under ATCC Accession Number X2.

2. A Lactuca sativa plant produced by growing the seed of claim 1.

3. A plant part from the plant of claim 2.

4. The plant part of claim 3, wherein said part is a head, a leaf, or a portion thereof.

5. The plant part of claim 4, wherein said part is a head.

6. A Lactuca sativa plant having all the physiological and morphological characteristics of the Lactuca sativa plant of claim 2.

7. A plant part from the plant of claim 6.

8. The plant part of claim 7, wherein said part is a head, a leaf, or a portion thereof.

9. The plant part of claim 8, wherein said part is a head.

10. An F₁ hybrid Lactuca sativa plant having ‘PS 1541’ as a parent wherein ‘PS 1541’ is grown from the seed of claim 1.

11. A pollen grain or an ovule of the plant of claim 2.

12. A tissue or cell culture produced from protoplasts or cells from the plant of claim 2, wherein said cells or protoplasts are produced from a plant part selected from the group consisting of root, root tip, meristematic cell, stem, hypocotyl, petiole, cotyledon, leaf, flower, anther, pollen, pistil, embryo, seed, and fruit.

13. A Lactuca sativa plant regenerated from the tissue or cell culture of claim 12, wherein the plant has all of the morphological and physiological characteristics of a Lactuca sativa plant produced by growing seed designated as ‘PS 1541’ having ATCC Accession Number X2.

14. A method of making Lactuca sativa seeds, said method comprising crossing the plant of claim 2 with another lettuce plant and harvesting seed therefrom.

15. The method of claim 14, wherein the method further comprises planting the harvested seed to grow F1 progeny.

16. The method of claim 15, wherein the method further comprises:
(a) selecting one or more of the F1 progeny for plants that have a desired trait to produce selected progeny plants;
(b) crossing the selected progeny plants with the plant of claim 2 to produce backcross progeny plants;
(c) selecting for backcross progeny plants that have the desired trait and all of the physiological and morphological characteristics of the plant of claim 2 when grown in the same environmental conditions to produce selected backcross progeny plants; and
(d) repeating steps (b) and (c) three or more times in succession to produce selected fourth or higher backcross progeny plants that comprise the desired trait and all of the physiological and morphological characteristics of the plant of claim 2 when grown in the same environmental conditions.

17. A method of producing a seed of a lettuce plant derived from the plant of claim 2, comprising crossing the plant of claim 2 with a second lettuce plant, whereby seed of a lettuce plant derived from the plant of claim 2 forms.

18. A derived lettuce plant produced by growing the seed of claim 17.

19. The method of claim 17, further comprising:
(a) crossing the derived lettuce plant with itself or a second lettuce plant to yield additional seed of the derived lettuce plant;
(b) growing the additional seed of the derived lettuce plant of step (a) to yield additional derived lettuce plants; and
(c) repeating the crossing and growing steps of (a) and (b) for an additional 3-10 generations to generate further derived lettuce plants.

20. A method of producing a lettuce leaf or head, said method comprising:
(a) growing the plant of claim 2 until it produces at least one leaf or head; and
(b) harvesting the leaf or head.
